(19) United States
(12) Patent Application Publication
(10) Pub. No.: US 2004/0199205 A1
(43) Pub. Date: Oct. 7, 2004

(54) Handle for forceps/tweezers and method and apparatus for designing the like

(76) Inventor: Stephen L. Tillim, Los Altos Hills, CA (US)

Correspondence Address: REED SMITH LLP, Suite 1400, 3110 Fairview Park Drive, Falls Church, VA, 22042, US

(21) Appl. No.: 10/829,346

(22) Filed: Apr. 22, 2004

Related U.S. Application Data
(63) Continuation-in-part of application No. 10/279,111, filed on Oct. 24, 2002.
(63) Continuation-in-part of application No. PCT/US02/33956, filed on Oct. 24, 2002.
(63) Continuation-in-part of application No. 10/420,872, filed on Apr. 23, 2003.
(63) Continuation-in-part of application No. PCT/US03/12648, filed on Apr. 23, 2003.

(51) Int. Cl. A61B 17/44

(52) U.S. Cl. 606/205

(57) ABSTRACT

Forceps/tweezers handles or apparatus for use with a hand include a proximal section, a middle section and a distal section. The proximal section engages one of a portion of radial or the ulnar side of the palmar surface of the hand without placing substantial pressure on a surface of the hand located over the carpal tunnel. The middle section connects with the proximal section and with the distal section and engages at least one the middle finger, ring finger or small finger of the hand. The distal section extends from the middle section and receives the thumb and at least of the index finger or middle finger of the hand. The distal section can include a working end and an implement can be attached to the working end of the distal end of the distal section.

### CLAIM FOR PRIORITY

This application claims the benefit of and is a Continuation-In-Part of U.S. application Ser. No. 10/279,111 filed on Oct. 24, 2002, the entire disclosure of which is incorporated herein by reference; this application also claims the benefit of and is a Continuation-In-Part of International Application No. PCT/US02/33956, filed on Oct. 24, 2002, published in English under PCT Article 21(2), the entire disclosure of which is incorporated herein by reference; this application claims the benefit of and is a Continuation-In-Part of U.S. application Ser. No. 10/420,872 filed on Apr. 23, 2003, the entire disclosure of which is incorporated herein by reference; this application also claims the benefit of and is a Continuation-In-Part of International Application No. PCT/US03/12648, filed on Apr. 23, 2003, believed not to be published in English under PCT Article 21(2), the entire disclosure of which is incorporated herein by reference; and this application further claims the benefit of U.S. Provisional Patent Application Serial No. 60/330,527 filed on Oct. 24, 2001, the entire disclosure of which is incorporated herein by reference.

### FIELD OF INVENTION

The present invention provides for forceps/tweezers handles or apparatus and method and apparatus for designing such forceps/tweezers handles or apparatus. The forceps/tweezers handles or apparatus are desirably designed to comfortably fit the hand when used. The present invention provides a forceps/tweezers handle or apparatus that desirably includes two mirror image blades, which meet or connect at one end. Forceps/tweezers handles or apparatus of the present invention can be used as a handle to assist the hand in pinching, gripping, holding, cutting and other functions. Forceps/tweezers handles or apparatus of the present invention can be used for a variety of surgical forceps, a variety of tweezers and a variety of tools and instruments.

### BACKGROUND OF THE INVENTION

Forceps and tweezers are common tools made in the shape of a stylus having mirror image blades connected at one end and open at the working end. Typically, forceps and tweezers are held close to their working ends by the thumb, index finger and middle finger like a pencil and rest on the fleshy space between the base of the thumb and the index finger. As used herein, as in anatomy, the term proximal is nearer and distal is further away on an extremity in relation to the torso. Similarly, in relation to the hand, typically the part of a forceps or tweezers resting on the portion of the hand between the base of the thumb and index finger is the proximal end, whereas the tips, or working ends, of forceps or tweezers can be referred to as the distal end.

Forceps and tweezers have blades or members enabling their tips to move closer for grasping and holding parts of various objects. This is done with a range of grip intensity by the opposing action of the thumb and the long fingers which manipulate the blades to move the tips of forceps or tweezers together. Fine, or light, pinch is produced by contracting the opponens muscle of the thumb and contracting the lumbrical muscles of the long fingers when the long fingers are extended. Contracting the short opponens muscle of the hand pulls the base of the thumb and contracting the small lumbrical muscles, located in the palm of the hand, pulls the bases of the long fingers. However, the function of gross pinch of forceps or tweezers is performed by the flexed thumb and long fingers under control of the long opponens muscle of the forearm and the deep flexor muscles of the forearm, which respectively pull the distal segment of the thumb and the distal segments of the long fingers.

Various factors can strain the muscles and joints of the hand and wrist when forceps and tweezers are used. Some factors causing strain include blade size and material. Typically, wider blades are easier to hold than narrow blades and generally require less muscle tension for pinching. The material used in fabricating and the method for connecting blades can affect spring-like properties of forceps or tweezers and can also affect the muscle force required to close them.

Another factor affecting muscle strain is the position of the hand when supporting forceps or tweezers. Typically, at rest a hand supports forceps or tweezers by the middle finger crossing underneath the blades and the fleshy portion of the hand located between the thumb and index finger. However, when forceps or tweezers are squeezed the opposing muscles of the thumb and the index finger contribute support, and these muscles must work harder.

Joint strain from grasping or pinching with forceps or tweezers can be related to the range and direction of motion in a joint and the anatomical position assumed by a hand and wrist. Some joints move mostly in one direction and have limited side to side motion. Other joints can move in more than one direction. In the latter type, for example, where the base of the thumb articulates with the wrist, the contact surface area is larger at the center of the joint than at the periphery. Therefore, greater surface area in the joint is available for contact when the thumb opposes the center of the tips of the long fingers than when the thumb opposes the index finger or small finger. However, when forceps or tweezers are squeezed between the index finger and thumb, contact is limited to the periphery of the joint at the base of the thumb and wrist and the joint can be strained.

Muscle strain is also affected whether the thumb opposes the index and middle fingers or the index finger alone, because the difference in the position of the thumb determines which portion, and which fascicles, of the opponens muscle contract. Muscles are built of groups of fascicles which are made up of groups of fibers. Force for pinching is greater when more muscle fascicles are available to contract. Likewise, more opponens fascicles are available when the thumb opposes the index and middle fingers than when the thumb opposes the index finger. This is because fewer fascicles are used in the radial side, compared to the center, of the opponens muscle to pinch common forceps or tweezers between the thumb and index finger. Also, strain and fatigue increase in the part of the muscle where fewer muscle fascicles are used for pinch. Therefore, a forceps or tweezers utilizing more muscle fascicles can desirably increase pinch strength and reduce muscle fatigue and stress.

Furthermore, using a greater number of muscles to perform a function produces greater strength. Pinching with the thumb opposing the space between the index finger and middle finger uses three muscles, one to pull the index finger, one to pull the middle finger and one muscle to pull the thumb. Pinching with the thumb against the index finger uses two muscles, one to pull the index finger and one to pull the thumb. Therefore, pinch forces are potentially greater when the thumb opposes both the index finger and the middle finger than when the thumb opposes the index finger alone because more muscle fascicles are potentially available for pinch strength.

Strain to the ligaments and joints in the hand can also occur from using a common forceps or tweezers because, like holding a stylus, the thumb and index finger tend to advance toward the tip of the common forceps or tweezers. As this happens, flexion becomes exaggerated at the distal interphalangeal joint (DIP) of the thumb and the middle interphalangeal (MIP) joint of the index finger, while the DIP joint of the index finger extends. Maintaining this position requires the forearm muscles to squeeze the ends of the fingers against the blades of the forceps or tweezers. This awkward position can strain finger and wrist joints and ligaments, especially if these joints have pre-existing damage. Furthermore, the tightened tendons from the forearm muscles holding such awkward position can transmit pressure within the carpal tunnel (CT) to the transverse carpal ligament (TCL) and to the median nerve, resulting in strain which can lead to median nerve irritation and carpal tunnel syndrome (CTS). Furthermore, this position can strain the muscles in the hand and forearm which can lead to repetitive strain syndrome.

Thus, frequent use of common forceps and tweezers often force hands into uncomfortable positions that can typically cause hand, wrist and forearm pain and lead to CTS. These problems have not been solved because common forceps or tweezers generally pinch with a stylus-type tool held between the thumb and index finger. A goal in designing forceps or tweezers handles or apparatus, as well as an objective of the method and apparatus of the present invention, is to promote the parts of the hand to work in harmony in performing a task, by maintaining the hand in a comfortable position while facilitating the hand and forearm muscles to work in concert. In addition, a goal in designing forceps or tweezers handles or apparatus, as well as an objective of the method and apparatus of the present invention, is promoting a reduction of the pinch strength typically required for holding an object. Furthermore, another goal in designing forceps or tweezers handles or apparatus, as well as an objective of the method and apparatus of the present invention, is to separate the functions of pinch and support performed while using forceps or tweezers. Thus, developing forceps or tweezers that position the thumb to oppose the space between the index finger and the middle finger can reduce the pinch strength required to hold objects, decrease muscle fatigue, reduce strain to joints and ligaments in the hand and wrist, and reduce hand pain.

Forceps and tweezers, such as surgical forceps and tweezers, generally fall into three common types. The first type has two side by side blade members hinged at one end and free working ends or tips at the other respective end. The working ends of blade members move toward each other and the tips come together to grasp and hold. The working ends of blade members of this first type of forceps and tweezers can cross distally at a hinge and close similar to scissors. The blade members of the second type of forceps and tweezers are oriented one on top of the other instead of side by side. In the second type, the handles of the forceps or tweezers extend perpendicular to the orientation of the blade members and typically have rings to engage the fingers. The blade members in the second type meet the handles at a hinge. Moving the rings on the handles moves a pivoting member to open or close for grasping or cutting tissue. A third type of forceps and tweezers uses a lever or slide to actuate a mechanism that opens and closes the jaws of an instrument.

The first type of forceps having side-by-side blades is of interest relating to the proposed handles or apparatus for forceps and tweezers of the present invention. Examples of the side-by-side blades of the first type of forceps or tweezers include those in U.S. Pat. Nos. 288,096, 987,095 and 2,540,255, which are fruit pickers. U.S. Pat. No. 5,893,877 illustrates a forceps or tweezers which is a microsurgical cup forceps. U.S. Pat. No. 5,002,561 illustrates a protective hand forceps. Further, U.S. Pat. No. 5,176,696 is related to handles for microsurgical instruments, with the handles in U.S. Pat. No. 5,176,696 opposing the thumb to the index finger and middle finger.

The problem with many of the above examples of common forceps or tweezers is that their design and operation does not take advantage of the greater pinch strength available from opposing the thumb to the index finger and middle finger, instead of opposing the thumb to the index finger, as well as not efficiently utilizing the palm of the hand to support the handle. In addition, the handles for common forceps and tweezers do not efficiently utilize the ring finger with or without the small finger to further hold and stabilize the handle of the forceps or tweezers. Therefore, what is needed is a forceps or tweezers that allows the hand to pinch with greater efficiency, improved stability and reduced joint and muscle strain.

### SUMMARY OF THE INVENTION

The present invention provides handles for forceps/tweezers and method and apparatus for designing such handles or apparatus.

Also, the design method and apparatus for forceps/tweezers handles or apparatus of the present invention includes embodiments and methods based on measurements made of the hand in a functional pinching position or Forceps Hand Position (FHP).

A method and apparatus for designing forceps/tweezers handles or apparatus and method and apparatus for designing such handles or apparatus of the present invention is provided and takes into consideration defined anatomical positions derived from the functional anatomy of a pinching hand. The methods of the present invention use lines with respect to measurements made in relation to the hand when the thumb opposes the space between the index and middle fingers. Apparatus, such as forceps and tweezers, produced from this method make efficient use of the hand.

An advantage of forceps/tweezers handles or apparatus and method and apparatus for designing such handles or apparatus of the present invention is that it promotes avoiding placing undue or substantial pressure on, or desirably avoids contact with, the area of the hand over the TCL. Therefore, the TCL is not compressed and substantial pressure is not transmitted to the contents of the CT region during pinching or using forceps/tweezers handles or apparatus of such design of the present invention.

Another advantage of forceps/tweezers handles or apparatus and method and apparatus for designing such handles or apparatus of the present invention is that the natural arcs of the fingers and palm are maintained providing a handle or apparatus that is more comfortable to hold.

Another advantage of forceps/tweezers handles or apparatus and method and apparatus for designing such handles or apparatus of the present invention is that more segments of the hand contact the handle providing a greater hand area to contact a handle or apparatus.

Another advantage of forceps/tweezers handles or apparatus and method and apparatus for designing such handles or apparatus of the present invention is that the arteries supplying the muscles in the hand are not compromised or distorted.

Another advantage of forceps/tweezers handles or apparatus and method and apparatus for designing such handles or apparatus of the present invention is that they do not compromise, compress or distort the nerves that go to the hand.

Another advantage of forceps/tweezers handles or apparatus and method and apparatus for designing such handles or apparatus of the present invention is that there is less compression, distortion or irritation of the median nerve by the superficial flexor tendons, which are closer to the TCL and the median nerve in the CT.

A significant advantage of the forceps/tweezers handles or apparatus and method and apparatus for designing such handles or apparatus of the present invention is that acute and chronic irritation, trauma and strain to the tendons, bursa, joints, hand muscles and median nerve are reduced.

It is an objective of the present invention to provide forceps/tweezers handles or apparatus and method and apparatus for designing such handles or apparatus of the present invention having greater contact with the supportive areas of the hand.

It is an objective of the present invention to provide forceps/tweezers handles or apparatus and method and apparatus for designing such handles or apparatus of the present invention to optimize use of the flexor hand muscles for the thumb and long fingers.

It is still another objective of the present invention to provide forceps/tweezers handles or apparatus and method and apparatus for designing such handles or apparatus of the present invention that utilize reduced grip effort as compared to a common forceps/tweezers.

It is still another objective of the present invention to provide forceps/tweezers handles or apparatus and method and apparatus for designing such handles or apparatus of the present invention of various sizes and shapes for various applications.

It is still another objective of the present invention to provide forceps/tweezers handles or apparatus and method and apparatus for designing such handles or apparatus of the present invention related to various hand sizes to accomplish the above and other objectives of the present invention.

According to another aspect of the present invention, forceps/tweezers handles or apparatus and method and apparatus for designing such handles or apparatus of the present invention can include those desirably having a curved shape, generally suggestive of a curved line similar to an arc, extending horizontally with the concave portion of the curved arc shape on the upper side of the handle or apparatus. Furthermore, such an arc-shape has a branching section near the middle of the arc extending from the convex side of the arc. The arc-shape for such a forceps/tweezers handle or apparatus includes duplicated sections each having three ends with the proximal ends of the respective duplicated sections being connected. The forceps/tweezers handles or apparatus including such features of the present invention will generally have the two arc-shaped portions joined or meeting at their proximal end to perform a forceps/tweezers open and close pinching function. However, in other embodiments of the handle or apparatus for the forceps or tweezers of the present invention can also include a single arc-shape with a branching section, such as can be adapted for various mechanisms and implements. The proximal section or sections of the arc-shaped apparatus or handle in embodiments of the present invention can meet and touch areas on the radial side of the palm of the hand. The extended or branching section or sections of the arc-shaped handle or apparatus of the present invention can meet and touch areas on the radial side and the palmar side of the ring finger of the hand, as well. The distal section or sections of the arc-shaped handle or apparatus of the present invention can extend from the connection of the proximal sections or sections of such arc-shaped handle or apparatus to a location near the tips of the thumb, index finger and middle finger when extended. In embodiments of the handle or apparatus of the present invention having two arc-shaped portions, the distal ends of each arc-shaped portion move toward the other and are brought into opposing relation by the movement of the thumb on one arc-shaped portion and the movement of the index finger and middle finger on the other arc-shaped portion. A variety of working ends can be attached to the distal members of such arc-shaped handles or apparatus of the present invention by various means and can be used to perform various functions, such as grasping, biting or cutting various objects. Also, a variety of mechanical or electronic implements can be attached to the arc-shaped handles or apparatus or the present invention.

It is another objective of the present invention to desirably provide forceps/tweezers handles or apparatus and method and apparatus for designing such handles or apparatus of the present invention of a curved arc shape that use a flexed ring finger to pull the handle or apparatus of the present invention toward the radial side of the palm of the hand when the hand is in the Forceps Hand Position (FHP).

According to another aspect of the present invention, forceps/tweezers handles or apparatus and method and apparatus for designing such handles or apparatus of the present invention can include those desirably having a generally “jogged” shape similar to a road that is offset to one side to avoid an obstruction and then continues in another straight path. Furthermore, such a “jogged” shape for such forceps/tweezers handle or apparatus can include duplicated sections with the proximal ends of the respective duplicated sections being connected. While embodiments of such forceps/tweezers handles or apparatus of the present invention including the feature of the two “jogged” shaped portions joined or meeting at their proximal end to enable a pinching function, such handle or apparatus for the forceps or tweezers can also be of a single “jogged” shape, that can be adapted for various mechanisms and implements. In such “jogged” shape embodiments, the proximal section or sections of each “jogged” shape can meet and touch at an area on the ulnar side of the palm of the hand. An offset or extended portion of such “jogged” shape embodiments of the handle or apparatus is generally located at the middle section or sections of each “jogged” shape for meeting and touching areas on the radial side and palmar side of the ring finger and areas on the palmar side of the small finger of the hand. The distal section or sections of such “jogged” shape embodiments of the handle or apparatus can extend from the offset or extended portion of the middle section or sections of the “jogged” shape handle or apparatus to a location near the tips of the thumb, index finger and middle finger. Also, the distal section or sections of such “jogged” shape embodiments of the handle or apparatus can contact the distal part of the thumb, index finger and middle finger of the hand. In embodiments of the handle or apparatus of the present invention having two, or a pair of, “jogged” shaped portions, the distal ends of each “jogged” shaped portion move toward the other and are brought into opposing relation by the movement of the thumb on one “jogged” shaped portion and the movement of at least one of the index finger and middle finger on the other “jogged” shaped portion. A variety of working ends can be attached to the distal members of such “jogged” shaped handles or apparatus of the present invention by various means and can be used to perform various functions, such as grasping, biting or cutting various objects. Also, a variety of mechanical or electronic implements can be attached to the “jogged” shaped handles or apparatus or the present invention.

An additional objective of the present invention is to desirably provide forceps/tweezers handles or apparatus and method and apparatus for designing such handles or apparatus of the present invention of a curved “jogged” shape that can contact the hypothenar muscle area between the horizontal crease on the ulnar side of the hand and the pisiform bone on the ulnar side of the hand.

It is an objective of the present invention to desirably provide forceps/tweezers handles or apparatus and method and apparatus for designing such handles or apparatus of the present invention of a curved “jogged” shape that position the handle or apparatus in the hand by having the ring and small fingers wrap around the middle section member or members of the handle or apparatus.

It is another objective of the present invention to desirably provide forceps/tweezers handles or apparatus and method and apparatus for designing such handles or apparatus of the present invention of a curved “jogged” shape that use either the flexed ring finger and small finger or the flexed middle finger, ring finger and small finger to pull the handle or apparatus of the present invention toward the ulnar side of the palm of the hand when the hand is in the Forceps Hand Position (FHP).

According to another aspect of the present invention, forceps/tweezers handles or apparatus and method and apparatus for designing such handles or apparatus of the present invention desirably provide for the thumb to oppose either or both the index and middle fingers.

According to a further aspect of the present invention, forceps/tweezers handles or apparatus and method and apparatus for designing such handles or apparatus of the present invention desirably provide greater stabilization when used with a hand.

Furthermore, forceps/tweezers handles or apparatus and method and apparatus for designing such handles or apparatus of the present invention desirably optimize the position for the joints of the thumb and either or both of the index finger and middle finger so the respective MIP and DIP joints cannot flex excessively.

Moreover, it is an objective of the present invention to desirably provide forceps/tweezers handles or apparatus and method and apparatus for designing such handles or apparatus of the present invention that prevent the handle or apparatus from slipping within the hand.

Also, an objective of the present invention is to desirably provide forceps/tweezers handles or apparatus and method and apparatus for designing such handles or apparatus of the present invention that use the flexed ring finger to lift the handle or apparatus as the flexed ring finger contacts the proximal portion of the distal section or sections of the handle or apparatus.

Therefore, the present invention desirably provides forceps/tweezers handles or apparatus for use with a hand, which include a proximal section, a middle section and a distal section. The proximal section is for engaging one of a portion of radial side of the palmar surface of the hand or the ulnar side of the palmar surface of the hand without placing substantial pressure on a surface of the hand located over the carpal tunnel. The middle section connects with the proximal section and with the distal section, with the middle section for engaging at least one of the middle finger, ring finger or small finger of the hand. The distal section extends from the middle section for receiving the thumb and at least one of the index finger or middle finger of the hand. Also, the distal section can include a working end, such as can be used for grasping, pinching or cutting. Further, an implement can be attached to the working end of the distal end of the distal section.

Additionally, a forceps/tweezers handle or apparatus of the present invention is desirably positioned within the hand without engaging a surface of the hand located over the carpal tunnel.

### BRIEF DESCRIPTION OF THE DRAWINGS

The foregoing and additional features and characteristics of the present invention will become more apparent from the following detailed description considered with reference to the accompanying drawings in which like reference numerals designate like elements and wherein:

FIG. 1A and FIG. 1B are views of the hand in the Forceps Hand Position (FHP). FIG. 1A illustrates the palmar surface view with the thumb, index finger and middle finger ending in the same plane. The tips of the ring finger and small finger also end on the same line. FIG. 1B is a view of the hand in the Forceps Hand Position (FHP) from the perspective of the radial side of the hand. FIG. 1B also illustrates the thumb, index finger and ring finger ending at the same plane and a centerline C1 extending along another plane from the horizontal crease to bisect the distance between the opposing thumb, index finger and ring finger.

FIG. 2 is a radial view of the hand in the Forceps Hand Position (FHP) illustrating Plane C. Plane C includes the centerline C1 of FIG. 1B and passes through the horizontal line on the radial side of the hand and through area M on the ulnar side of the hand.

FIG. 3A and FIG. 3B are views illustrating the first of two styles of forceps/tweezers handles or apparatus of the present invention. FIG. 3A is three-dimensional view and FIG. 3B is a side or profile view.

FIG. 4A and FIG. 4B are views illustrating the second of two styles of forceps/tweezers handles or apparatus of the present invention. FIG. 4A is three-dimensional view and FIG. 4B is a side or profile view.

FIG. 5A and FIG. 5B are views illustrating a hand in the Forceps Hand Position (FHP) with the hand holding forceps/tweezers handles or apparatus of the present invention. FIG. 5A is a palmar view of the hand holding the arc-shaped type of forceps/tweezers handle or apparatus and FIG. 5B is a palmar view of the hand holding a “jogged” shaped forceps/tweezers handle or apparatus.

FIG. 6A and FIG. 6B are radial views illustrating a hand in the Forceps Hand Position (FHP) with the hand holding the corresponding forceps/tweezers handles or apparatus of the present invention. FIG. 6A is a radial view of the hand holding an arc-shaped forceps/tweezers handle or apparatus illustrating the centerline C1 in Plane C of the arc-shaped forceps/tweezers or apparatus in relation to the horizontal crease on the radial side of the hand. FIG. 6B is a radial view of the hand holding a “jogged” shaped forceps/tweezers handle or apparatus illustrating the centerline C1 in Plane C of the arc-shaped forceps/tweezers or apparatus in relation to an area on the ulnar side of the hand.

FIG. 7A and FIG. 7B illustrate a rectangular measuring device used to measure the hand for determining measurements and locations of lines related to the measurements for producing sizes for a forceps/tweezers handle or apparatus of the present invention. FIGS. 7C and 7C1 illustrate an imaging system with a hand in the Forceps Hand Position (FHP) on a grid system for the purpose of making digital measurements for producing sizes for a forceps/tweezers handle or apparatus of the present invention. FIG. 7D and FIG. 7E illustrate the arrangement, measurements and locations of such lines and sections used to produce a handle or apparatus of the present invention. Specifically, FIG. 7F illustrates the lines and sections that can be used to produce an arc-shaped forceps/tweezers handle or apparatus and FIG. 7G illustrates the lines and sections that can be used to produce a “jogged” shape forceps/tweezers handle or apparatus of the present invention.

FIGS. 8A, 8B and 8C illustrate variations of forceps/tweezers handles or apparatus of the present invention where the distal end is related to either the thumb opposing at least one of the index finger or the middle finger. FIG. 8A illustrates a variation at the distal end of the arc-shaped forceps/tweezers handle or apparatus. FIG. 8B illustrates a variation where the middle finger, ring finger and small finger hold the middle sections of the “jogged” shape forceps/tweezers handle or apparatus. FIG. 8C illustrates a variation where the ring finger and small finger hold the middle sections of the “jogged” shape forceps/tweezers handle or apparatus.

FIGS. 9A, 9B and 9C illustrate palmar views of a hand holding variations of the forceps/tweezers handle or apparatus of the present invention illustrated in FIGS. 8A, 8B and 8C.

FIGS. 10A through 10E illustrate additions, offsets or extensions in the corresponding middle sections to augment holding of forceps/tweezers handles or apparatus of the present invention. FIGS. 10A and 10D illustrate scalloped distal surfaces of the middle sections. FIGS. 10B and 10E illustrate generally flat distal surfaces and FIG. 10C illustrates rings at the middle sections.

FIGS. 11A and 11B illustrate extenders added to the arc-shaped and “jogged” shape embodiments of forceps/tweezers handles or apparatus of the present invention to adjust forceps/tweezers handles or apparatus of the present invention for a plurality of hand sizes.

FIGS. 12A through 12D illustrate an elastic means, such as a spring, to keep the forceps/tweezers handles or apparatus in an open position.

FIGS. 13A through 13C illustrate additions to forceps/tweezers handles or apparatus of the present invention near the distal end of a handle or apparatus. FIG. 13A illustrates a clamp to maintain a handle or apparatus in a range from a fully open position to a fully closed position. FIGS. 13B and 13C illustrates finger guide members to receive the ends of corresponding fingers in a handle or apparatus.

FIG. 14 illustrates a connection means at the distal end of a handle or apparatus of the present invention for connecting various implements to the handle or apparatus.

FIGS. 15A through 15D illustrate working ends or implements attached to forceps/tweezers handles or apparatus of the present invention, with FIGS. 15A and 15C being a microscissors respectively attached to an arc-shaped and “jogged” shape forceps/tweezers handle or apparatus and with FIGS. 15B and 15D being a reverse tweezers respectively attached to an arc-shaped and “jogged” shape forceps/ tweezers handle or apparatus.

FIGS. 16A, 16B, 16C and 16D illustrate various embodiments of handles or apparatus of the present invention that can have devices connecting or integrated with a handle or apparatus. FIG. 16A illustrates an arc-shape unitary handle or apparatus of the present invention. FIG. 16B illustrates an “jogged” shape unitary handle or apparatus of the present invention. FIG. 16C illustrates an embodiment of a generally unitary arc-shaped handle or apparatus of the present invention that incorporates a motor driving means for rotation or movement of a working end or an implement. FIG. 16D illustrates an embodiment of a generally unitary “jogged” shape handle or apparatus of the present invention that incorporates a motor driving means for rotation or movement of a working end or an implement.

FIGS. 17A and 17B illustrate embodiments of implements with an arc-shaped unitary handle or apparatus and a “jogged” shape unitary handle or apparatus of the present invention with an attachment mechanism, with the attachment mechanism illustrated in FIGS. 17A and 17B being a hinge member arranged to allow angular movement of the working implement in relation to the handle or apparatus or allow angular movement of the handle or apparatus in relation to the working implement.

FIGS. 18A through 18E illustrate embodiments of mechanisms to change the direction and orientation of pinch with respect to a handle or apparatus of the present invention, such as from a side-to-side horizontal direction to an up and down vertical direction in relation to a handle.

FIGS. 19A, 19B, 19C and 19D illustrate a spring loaded mechanism, such as for a surgical scalpel guard, integrated with a handle or apparatus of the present invention to provide for retraction and extension of an implement for use with a handle.

### DESCRIPTION OF THE PREFERRED EMBODIMENTS

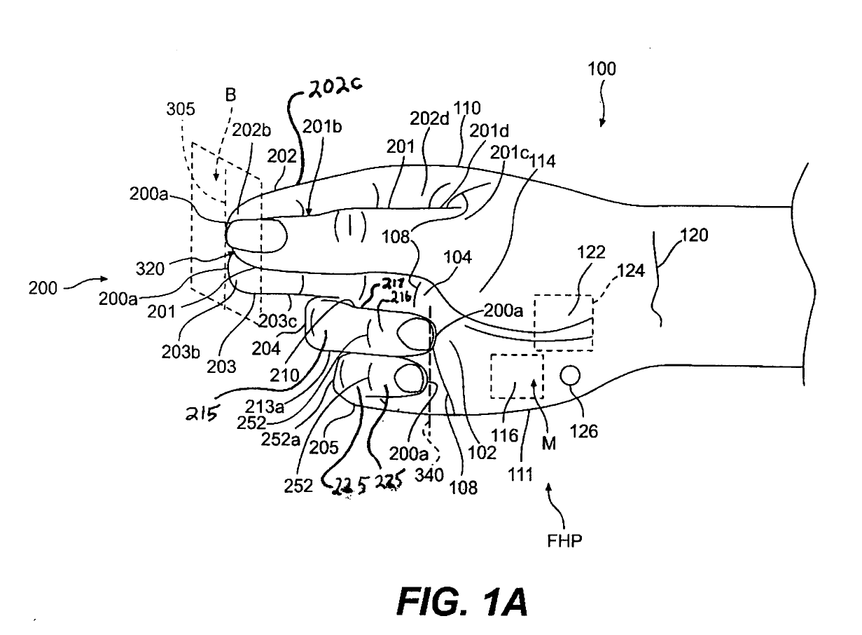

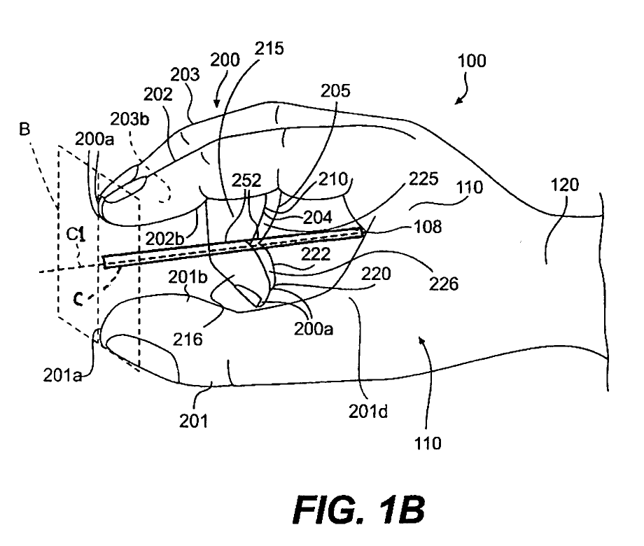

In order to more clearly and concisely describe the subject matter of the present invention, the following definition for the Forceps Hand Position (FHP) is intended to provide guidance as to the meanings of specific terms used in the following written description. In addition, it is to be understood that the phraseology or terminology employed herein is for the purpose of description and not to be construed in a limiting sense. The following discussion relates to areas of the hand in relation to the present invention with reference to FIGS. 1 through 6B.

Forceps Hand Position (FHP)

FIG. 1A and FIG. 1B illustrate a hand 100 in the Forceps Hand Position (FHP). In the Forceps Hand Position (FHP) the thumb 201, index finger 202 and middle finger 203 are partially extended and the tip 201a of the thumb 201 opposes the space 320 between the tip 200a of the index finger 202 and the tip 200a of the middle finger 203. Furthermore, as shown by the dashed line 305, when the hand is in the Forceps Hand Position (FHP) the tip 201a of the thumb 201 is in substantial alignment with the tip 200a of the index finger 202 and the tip 200a of the middle finger 203. In addition, when the hand is in the Forceps Hand Position (FHP), the tip 200a of the ring finger 204 and the tip 200a of the small finger 205 are adjacent to each other and end at line 340.

Continuing with reference to FIGS. 1A, the horizontal crease 108 crosses the palm 102 of the hand 100 and is hidden by the base 201d of the thumb 201 until the horizontal crease 108 reaches the radial side 110 of the hand 100. The location of the horizontal crease 108 at the radial side 110 of the hand 100 is also illustrated in FIG. 1B. Furthermore, as illustrated in FIG. 1A, when the hand 100 is in the Forceps Hand Position (FHP) the palmar arch 104 is concave and maintains an arcuate shape thereby preserving the neutral or resting position of the palm 102 of the hand 100.

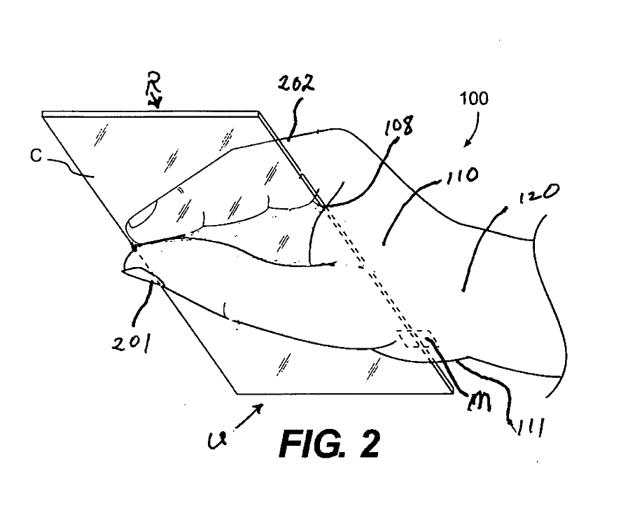

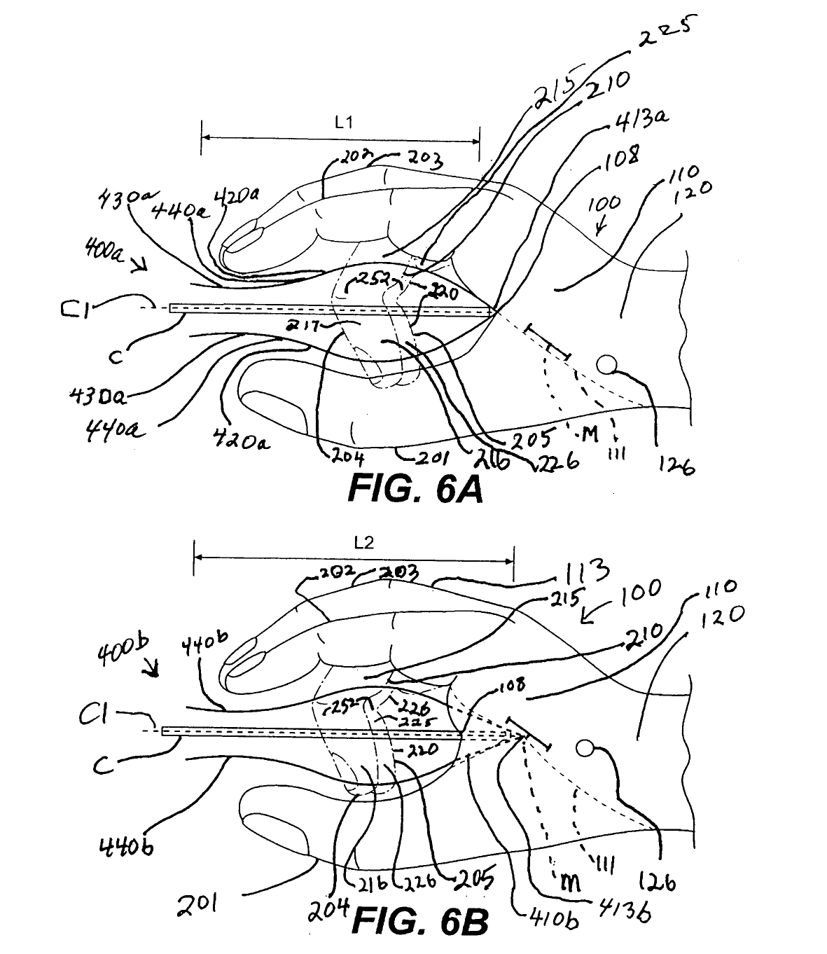

FIGS. 1B, 2, 6A and 6B illustrate Plane C, which bisects the space made between the thumb 201 and the index finger 202 and middle finger 203 of a hand 100 in the Forceps Hand Position (FHP). Plane C passes through the radial side 110 of the hand 100 and the ulnar side 111 of the hand 100 at the horizontal crease 108 on the radial side 110 of the hand 100 and area M on the ulnar side 111 of the hand 100, which is approximately half way between the horizontal crease 108 and the pisiform bone 126 of the wrist 120. Plane C also passes through the DIP joints 252 of the ring finger 204 and small finger 205. Furthermore, as illustrated in FIG. 2, the radial edge R of Plane C slants toward the radial side 110 of a hand 100 and the ulnar edge U of Plane C slants away from the ulnar side 111 of a hand when the hand is in the Forceps Hand Position (FHP) and viewed from the distal direction, i.e. facing the space between the thumb and index finger. Plane C, as illustrated in FIGS. 6A and 6B, substantially concurs, or is in substantial alignment, with the centerline C1 of a handle or apparatus of the present invention

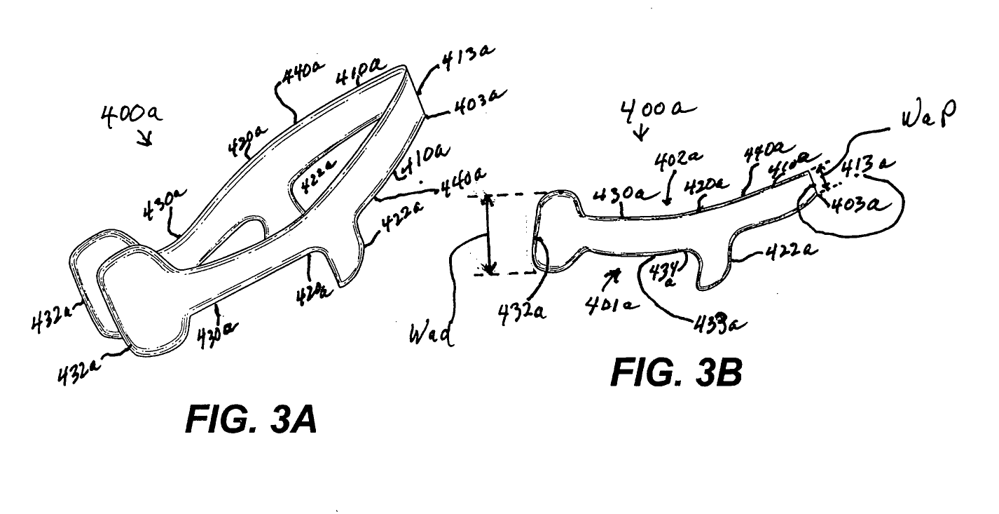

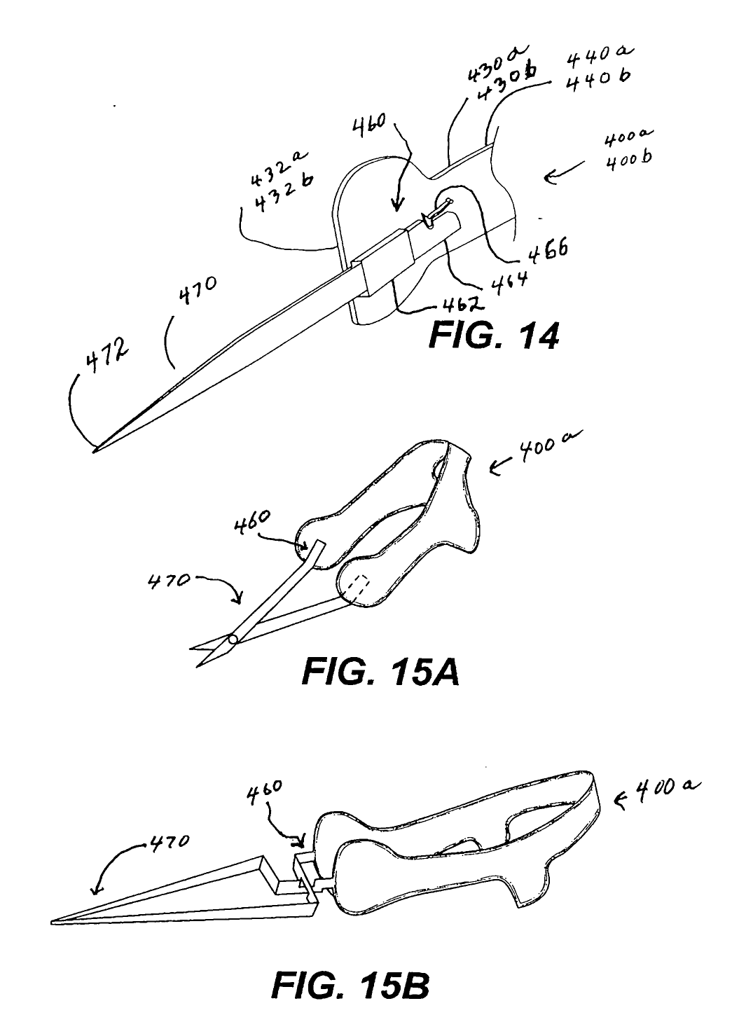

FIGS. 3A, 3B, 4A, 4B, 5A and 5B illustrate two embodiments of the forceps/tweezers handles or apparatus 400a, 400b of the present invention. Embodiments of the forceps/tweezers handles or apparatus 400a and 400b of the present invention have respective proximal sections 410a, 410b, middle sections 420a, 420b and distal sections 430a, 430b. The proximal sections 410a, 410b and the middle sections 420a, 420b of the forceps/tweezers handles or apparatus 400a and 400b of the present invention are supported by the hand 100. The distal sections 430a, 430b of the forceps/tweezers handles or apparatus 400a and 400b of the present invention are typically not supported by the hand 100. The embodiments of the forceps/tweezers handles or apparatus 400a and 400b of the present invention have two opposing blades 440a and 440b. The opposing blades 440a, 440b of the forceps/tweezers handles or apparatus 400a and 400b of the present invention can be mirror images of the other. The opposing blades 440a, 440b have corresponding proximal sections 410a, 410b, middle sections 420a, 420b and distal sections 430a, 430b. The distal sections 430a, 430b of the opposing blades 440a, 440b can perform grasp, pinch and other mechanical actions related to the opposing movement of the distal pad 201b of the thumb 201 toward the distal pad 202b of the index finger 202 and the distal pad 203b of the middle finger 203. As illustrated in FIGS. 14, 15A, 15B, 16C, 16D, 17A and 17B, the distal ends 432a, 432b of the distal sections 430a, 430b of each opposing blades 440a, 440b of the forceps/tweezers handles 400a and 400b can have working ends 470 that are integrated or that include various connecting means 460 to allow multiple, varied implements to performing various suitable tasks or functions, such as grasping, pinching, cutting, rotating or other electrical or mechanical functions.

The forceps/tweezers handle or apparatus 400a of the present invention illustrated in FIGS. 3A and 3B has an arc shape with the concave side 402a at the top and an extension from the convex side 401 at the bottom. Each opposing blade 440a of the forceps/tweezers handle 400a has a corresponding proximal section 410a, middle section 420a and distal section 430a. The proximal sections 410a of each blade 440a meet and are connected by a radial hinge 403a at the proximal end 413a of the forceps/tweezers handle 400a. The convex sides 401a have a corresponding extension 422a from a corresponding middle section 420a of the forceps/tweezers handle or apparatus 400a. The distal sections 430a each continue from the corresponding middle section 420a of the forceps/tweezers handle or apparatus 400a of the present invention. Also, each distal section 430a has a corresponding distal end 432a.

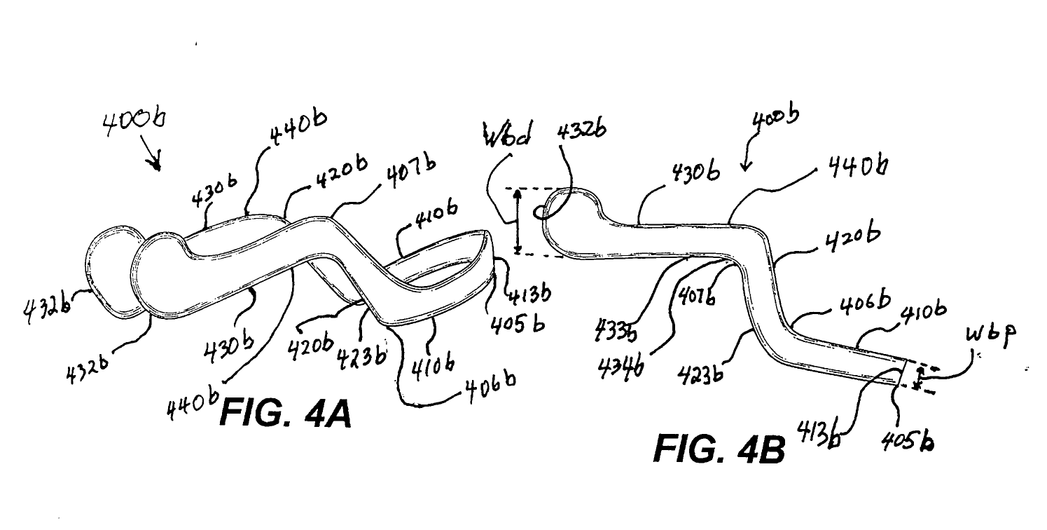

As illustrated in FIGS. 4A and 4B the forceps/tweezers handle or apparatus 400b of the present invention forms a shape of a configuration similar to a jog in the road with a straight entrance and a straight exit. Each opposing blade 440b of the forceps/tweezers handle or apparatus 400b has a corresponding proximal section 410b, middle section 420b and distal section 430b. The opposing blades 440b meet and connect at an ulnar hinge 405b at the proximal end 413b of the proximal sections 410b of the forceps/tweezers handle or apparatus 400b. The proximal sections 410b each meet the corresponding middle section 420b at a corresponding proximal curve 406b. The middle sections 420b each then respectively continue into the corresponding distal section 430b at the corresponding distal curve 407b. The distal sections 430b each respectively continue from the corresponding distal curve 407b of the corresponding middle sections 420b of the forceps/tweezers handle or apparatus 400b of the present invention. The distal sections 430b have corresponding distal ends 432b.

The hinges 403a and 405b at the respective proximal ends 413a, 413b of the respective forceps/tweezers handles or apparatus 400a and 400b can be made such that one opposing blade 440a, 440b is continuous or integrally formed into the other opposing blade 440a, 440b. Hinges 403a, 405b can also be made of a mechanical connection means, such as a hinge arrangement. The widths Wap, Wbp of the proximal ends 413a, 413b of the proximal sections 410a, 410b of the forceps/tweezers handles or apparatus 400a and 400b approximate the width of base 202d of the index finger 202. The width Wad, Wbd of the distal ends 432a, 432b of the distal sections 430a, 430b approximate the combined width of the distal pad 202b of the index finger 202 and the distal pad 203b of the middle finger 203.

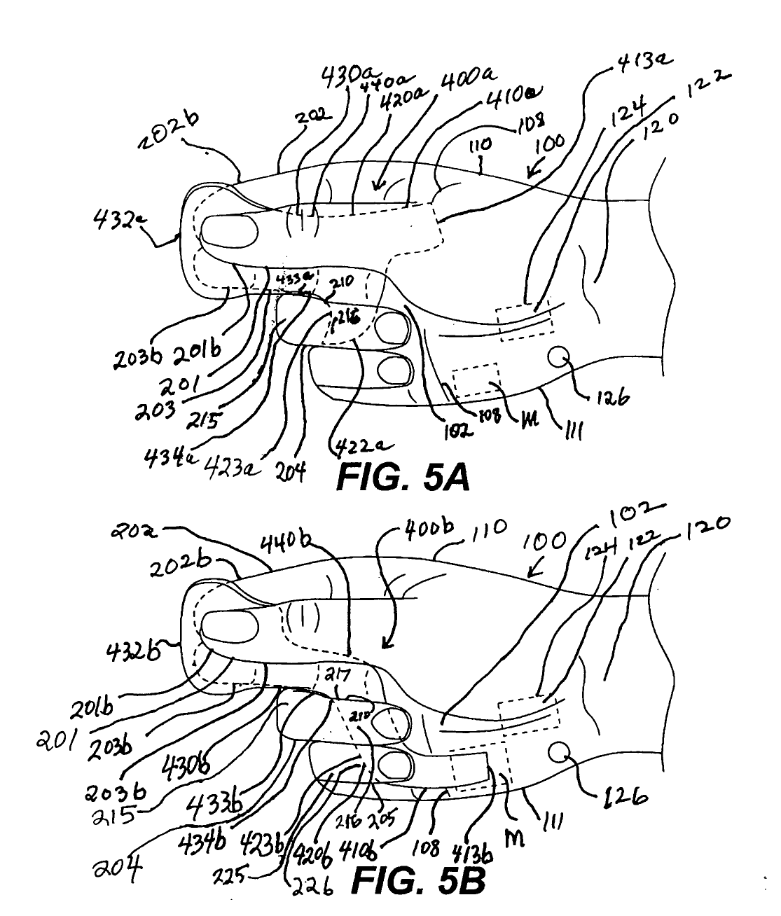

As illustrated in FIG. 5A, the proximal end 413a of the proximal sections 410a of the forceps/tweezers handle or apparatus 400a can be consistent with the corresponding surface of the palm 102 in the area near the horizontal line 108 at the radial side 110 of the hand 100. As illustrated in FIG. 5B, The proximal end 413b of the proximal sections 410b of the forceps/tweezers handle or apparatus 400b can be consistent with the corresponding surface of the palm 102 in area M at the ulnar side 111 of the hand 100. The shape of the proximal ends 413a, 413b of forceps/tweezers handle or apparatus 400a and 400b can be of a suitable shape, such as flat, rounded or angled.

FIGS. 5A, 5B, 6A, and 6B illustrate a hand 100 in the Forceps Hand Position (FHP) with the hand 100 holding forceps/tweezers handles or apparatus of the present invention. FIGS. 5A and 5B are palmar views of the hand 100 respectively holding forceps/tweezers handles or apparatus 400a and 400b. FIG. 6A is a radial view of the hand 100 holding forceps/tweezers handle or apparatus 400a, and FIG. 6B is a radial view of the hand 100 holding forceps/tweezers handle or apparatus 400b.

With reference to FIGS. 1A, 1B, 2B, 5A and 6A, desirably the horizontal crease 108 on the radial side 110 of the hand 100 contacts the proximal end 413a of each opposing blade 440a of the forceps/tweezers handle or apparatus 400a of the present invention. Continuing with reference to FIG. 5A and 6A, the palmar surface 210 of the middle phalange 215 of the ring finger 204 and the palmar surface 210 of the distal phalange 216 of the ring finger 204 contact the distal surface 423a of the corresponding extension 422a of the middle sections 420a of the forceps/tweezers handle or apparatus 400a of the present invention. In addition, the radial side 217 of the middle phalange 215 of the ring finger 204 and the radial side 217 of the distal phalange 216 of the ring finger 204 contact an inferior surface 434a the corresponding proximal area 433a of the distal sections 430a of the forceps/tweezers handle or apparatus 400a. Furthermore, the distal pad 201b of the thumb 201 contacts the distal end 432a of the distal section 430a of one opposing blade 440a of the forceps/tweezers handle or apparatus 400a of the present invention, and at least one of the distal pad 202b of the index finger 202 and the distal pad 203b of the middle finger 203 contacts the distal section 430a of the mirror image other opposing blade 440a of the forceps/tweezers handle or apparatus 400a of the present invention.

In addition, with reference to FIGS. 1A, 1B, 5B and 6B, the proximal end 413b of the corresponding proximal section 410b of each opposing blade 440b of the forceps/tweezers handle or apparatus 400b of the present invention contacts the ulnar side 111 of the palm 102 of the hand 100 at area M between the horizontal crease 108 and the pisiform bone 126 of the wrist 120. Further, the palmar surface 210 of the middle phalange 215 of the ring finger 204 and the palmar surface 210 of the distal phalange 216 of the ring finger 204 contact the corresponding distal surface 423b of the middle sections 420b of the forceps/tweezers handle 400b of the present invention. In addition, the radial side 110 of the middle phalange 215 of the ring finger 204 and the radial side 217 of the distal phalange 216 of the ring finger 204 contact an inferior surface 434b of the corresponding proximal area 433b of the distal sections 430b of the forceps/tweezers handle or apparatus 400b. Furthermore, the palmar surface 220 of the middle phalange 225 of the small finger 205 and the palmar surface 220 of the distal phalange 226 of the small finger 205 contact the corresponding distal surface 423b of the middle sections 420b of the opposing blades 440b of the forceps/tweezers handle or apparatus 400b of the present invention.

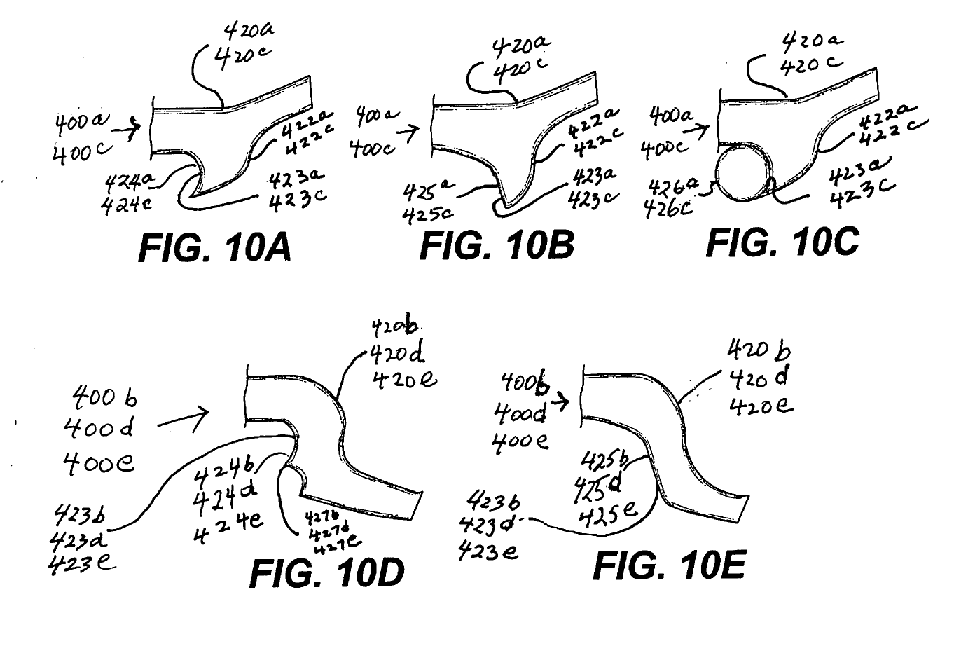

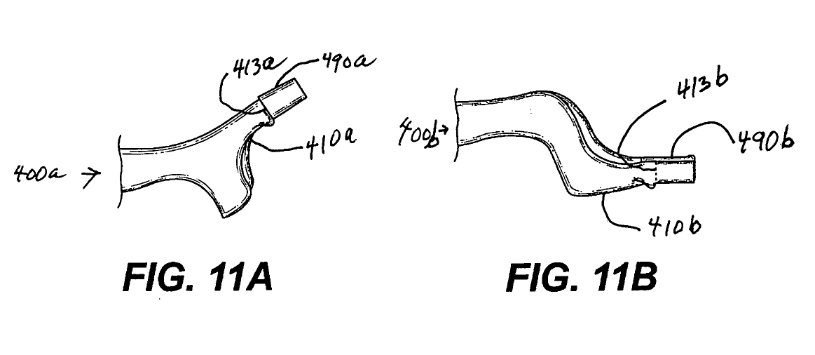

FIGS. 6A and 6B are radial views of a hand 100 holding a forceps/tweezers handle or apparatus of the present invention when the hand 100 is in the Forceps Hand Position (FHP) as viewed along Plane C. FIG. 6A is a radial view of a hand 100 holding forceps/tweezers handle or apparatus 400a of the present invention, whereas FIG. 6B is a radial view of a hand 100 holding forceps/tweezers handle or apparatus 400b of the present invention. FIGS. 6A and 6B illustrate superimposed views of forceps/tweezers handle or apparatus 400a and forceps/tweezers handle or apparatus 400b in relation to a hand. However, as illustrated in FIGS. 6A and 6B, Plane C, as discussed in relation to FIG. 11B and 2B, slants toward the radial side 110 of the hand 100. The forceps/tweezers handles or apparatus 400a and 400b appear to be a superimposed because the position of the hand 100 as illustrated in FIGS. 6A and 6B is pronated, i.e. the thumb and forearm rotate toward the body, to bring area M into view on the ulnar side 111 of the hand 100. Such rotation gives greater exposure to the dorsal (outer) side 113 of the middle finger 203 as illustrated in FIGS. 6A and 6B. Furthermore, the views illustrated in FIGS. 6A and 6B show the length L1 of forceps/tweezers handle or apparatus 400a is less than the length L2 of forceps/tweezers handle or apparatus 400b in relation to the thumb 201, index finger 202 and middle finger 203 of the hand 100 in an extended position, such as in the Forceps Hand Position (FHP).

As illustrated in FIGS. 5A and 6A, a hand 100 desirably supports the forceps/tweezers handle or apparatus 400a of the present invention, at three contact locations. The first support location is where the radial side 110 of the horizontal crease 108 of the hand 100 contacts the proximal end 413a of the proximal section 410a of each opposing blade 440a of the forceps/tweezers handle or apparatus of the present invention. The second support location is where the palmar surface 210 of the middle phalange 215 of the ring finger 204 and the palmar surface 210 of the distal phalange 216 of the ring finger contact the corresponding distal surfaces 423a of the extensions 422a of the respective middle sections 420a of the forceps/tweezers handle or apparatus 400a of the present invention. The third support location is where the radial side 217 of the middle phalange 215 of the ring finger 204 and the radial side 217 of the distal phalange 216 of the ring finger 204 contact the corresponding inferior surfaces 434a of the proximal areas 433a of the respective distal sections 430a of the forceps/tweezers handle or apparatus 400a.

Alternately, as illustrated in FIGS. 5B and 6B, a hand 100 desirably supports the forceps/tweezers handle or apparatus 400b of the present invention at four contact locations. The first support location can be where the ulnar side 111 of the horizontal crease 108 of the hand 100 contacts the corresponding proximal end 413b of the proximal section 410b of each opposing blade 440b of the forceps/tweezers handle or apparatus 400b of the present invention. However, the optimal second support location, as illustrated in FIG. 5B, is where the proximal end 413b of the proximal section 410b of each opposing blade 440a of the forceps/tweezers handle 400 of the present invention contacts area M located between the ulnar side 111 of the horizontal crease 108 and the pisiform bone 126 of the wrist 120 on the ulnar side 111 of the hand 100. The second support location is where the palmar surface 210 of the middle phalange 215 of the ring finger 204 and the palmar surface 210 of the distal phalange 216 of the ring finger respectively contact the corresponding distal surface 423b of the middle sections 420b of the opposing blades 440b of the forceps/tweezers handle or apparatus 400b of the present invention. The third support location is on the radial side 217 of the middle phalange 215 of the ring finger 204 and on the radial side 217 of the distal phalange 216 of the ring finger 204 which respectively contact the corresponding inferior surface 434b of the proximal areas 433b of the distal sections 430b of the forceps/tweezers handle or apparatus 400b. The fourth support location is where the palmar surface 220 of the middle phalange 225 of the small finger 205 and the palmar surface 220 of the distal phalange 226 of the small finger 205 respectively contact the corresponding distal surface 423b of the middle sections 420b of the respective opposing blades 440b of the forceps/tweezers handle or apparatus 400b of the present invention.

As illustrated in FIGS. 5A and 6A, support and stabilization within the hand 100 for a handle or apparatus of the present invention, such as the forceps/tweezers handle or apparatus 400a of the present invention, is enhanced by the deep flexor forearm muscle contracting the distal phalange 216 of the ring finger 204 and the superficial flexor forearm muscle contracting the middle phalange 215 of the ring finger 204. Such contraction pulls the proximal end 413a of the proximal sections 410a of the forceps/tweezers handle or apparatus 400a of the present invention against the horizontal crease 108 of the palm 102 at the radial side 110 of the hand 100. Support for lifting objects held by a handle or apparatus of the present invention, such as the forceps/tweezers handle or apparatus 400a of the present invention, by the hand 100 is enhanced by contact at the radial side 217 of the ring finger 204 with the corresponding inferior surface 434a of the proximal areas 433a of the respective distal sections 430a of the forceps/tweezers handle or apparatus 400a.

As illustrated in FIGS. 5B and 6B, support and stabilization within the hand 100 for the forceps/tweezers handle or apparatus 400b of the present invention, is enhanced by the deep flexor forearm muscle contracting the distal phalange 216 of the ring finger 204, and by the superficial flexor forearm muscle contracting the middle phalange 215 of the ring finger 204, and by the deep flexor forearm muscle contracting the distal phalange 226 of the small finger 205, and by the superficial flexor forearm muscle contracting the middle phalange 225 of the small finger 205 on the corresponding distal surface 423b of the middle sections 420b of the respective opposing blades 440b. Such contraction pulls the forceps/tweezers handle or apparatus 400b of the present invention against a location within area M of the palm 102 at the ulnar side 111 of the hand 100. Support for lifting objects held by a handle or apparatus of the present invention, such as the forceps/tweezers handle or apparatus 400b of the present invention, by the hand 100 is enhanced by contact at the radial side 217 of the ring finger 204 with the corresponding distal surface 423b of the middle sections 420b of the respective opposing blades 440b of the forceps/tweezers handle or apparatus 400b of the present invention.

In reference to FIGS. 5A and 6A, pinch, as a function of a forceps or tweezers, is performed when the distal ends 432a, 432b of the distal sections 430a, 430b of the opposing blades 440a 440b of forceps/tweezers handles or apparatus 400a and 400b occurs by the opposing movement of the thumb 201 and at least one of the index finger and middle finger. However, when closing a common forceps or tweezers, the thumb 201 and index finger 202 contribute to its support while simultaneously opposing each other, which can lead to strain and fatigue. On the other hand, when using the forceps/tweezers handles or apparatus 400a and 400b of the present invention, the thumb 201, index finger 202 and middle finger 203 are primarily involved with pinch and not support. Therefore, use of the forceps/tweezers handles or apparatus 400a and 400b of the present invention can reduce strain on the muscles flexing the thumb 201, index finger 202 and middle finger 203 when they are used to perform fine or gross pinch. Furthermore, as illustrated in FIGS. 5A through 6B, the forceps/tweezers handles or apparatus 400a and 400b promote the thumb 201, index finger 202 and middle finger 203 to be placed in a relaxed extended position, which also reduces tension on the joints of the hand 100. In addition, as illustrated in FIGS. 5A and 5B, the proximal ends 413a, 413b of the proximal sections 410a, 410b of forceps/tweezers handles or apparatus 400a and 400b touch the palm 102 of the hand 100 in such a way as to not place substantial or undue pressure on the contents of the CT area 124, the TCL 122 or the nerves or blood vessels supplying the hand 100. Also, another feature of forceps/tweezers handles or apparatus 400a and 400b is that such forceps/tweezers handles or apparatus can be used with a right hand 100 or a left hand 100.

Hand Measurements

A goal in developing the proposed forceps/tweezers handles or apparatus of present invention, such as forceps/tweezers handles or apparatus 400a and 400b, is to produce forceps/tweezers handles or apparatus that fit hands. It is possible to develop one size for forceps/tweezers handles or apparatus of the present invention, such as forceps/tweezers handles or apparatus 400a and 400b, to span many hands. However, holding either forceps/tweezers handles or apparatus 400a or 400b of the present invention will be more comfortable, require less muscular effort and have greater stability if made in multiple sizes. The shoe industry recognizes that feet have a range of lengths and widths. The same is true of hands. The length from wrist 120 to the tips 200a of the long fingers 200 and width from the radial side 110 of the hand 100 to the ulnar side 111 of the hand 100 vary such that hands can be short and long, short and narrow, long and wide and long and narrow. In general, male hands are longer and wider than female hands. Therefore, sizes for forceps/tweezers handles or apparatus of the present invention, such as forceps/tweezers handles or apparatus 400a and 400b, can be determined and can desirably be based upon measurements taken with the hand in the Forceps Hand Position (FHP) as illustrated in FIG. 1A and FIG. 1B.

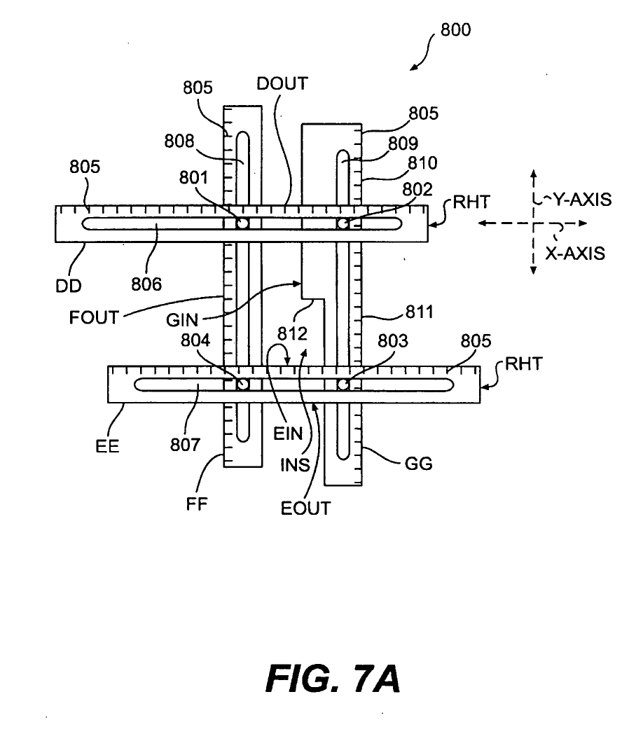

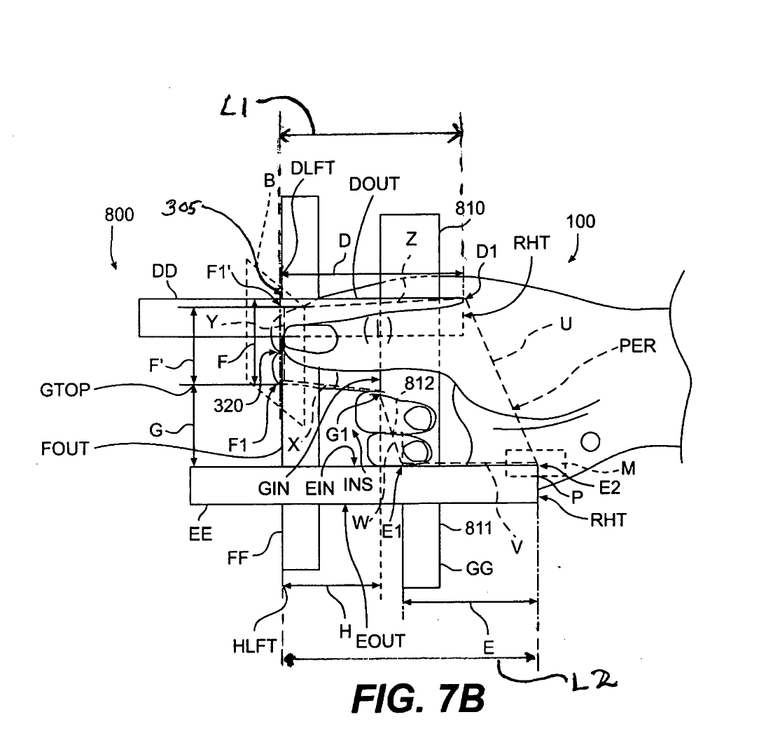

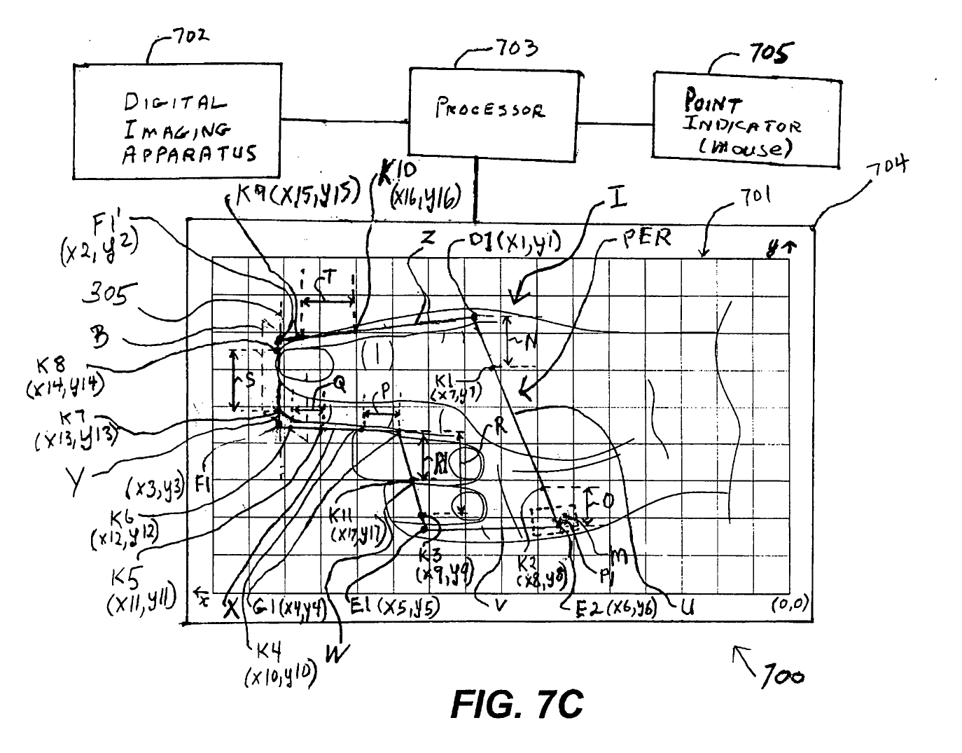

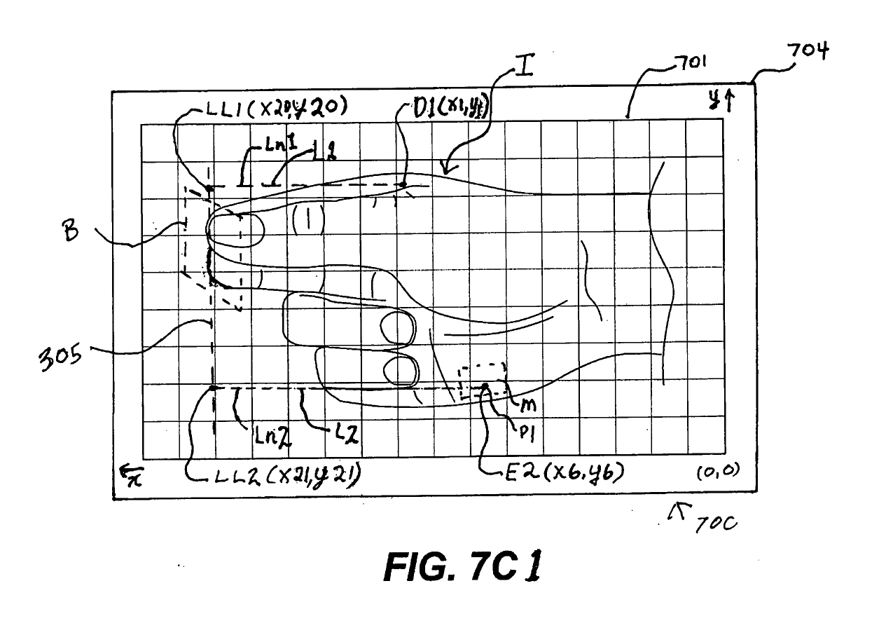

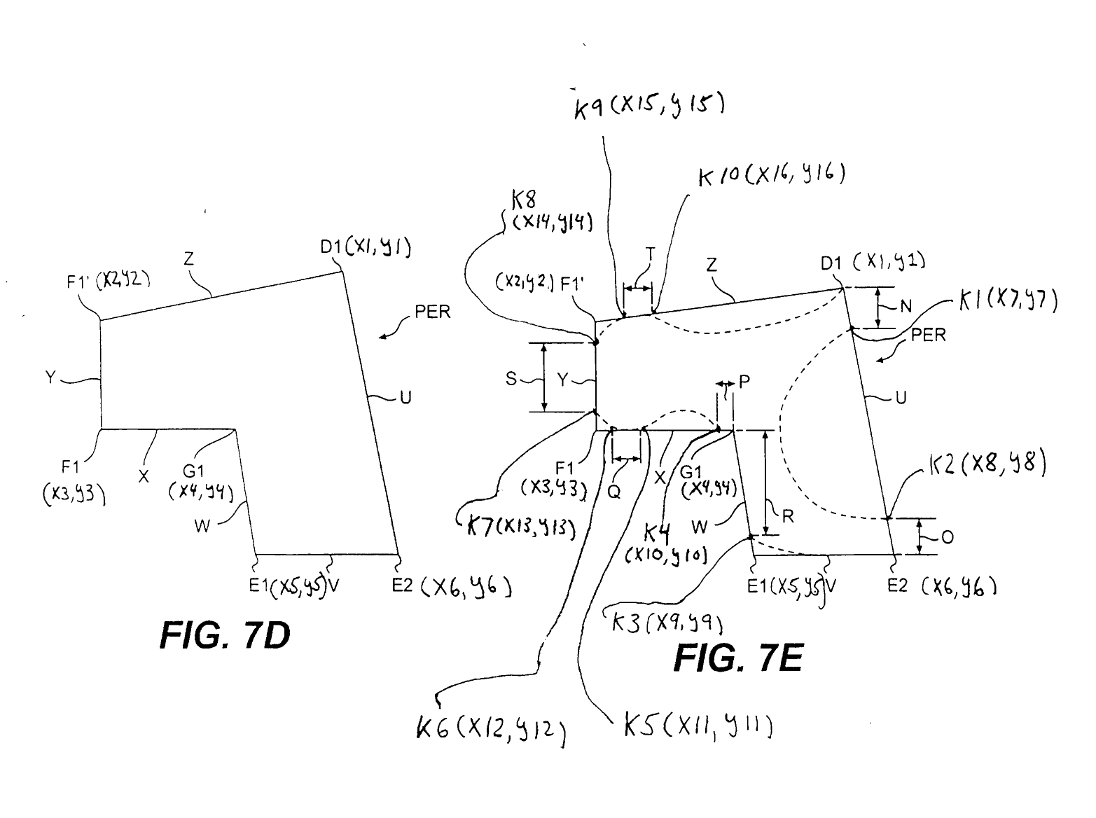

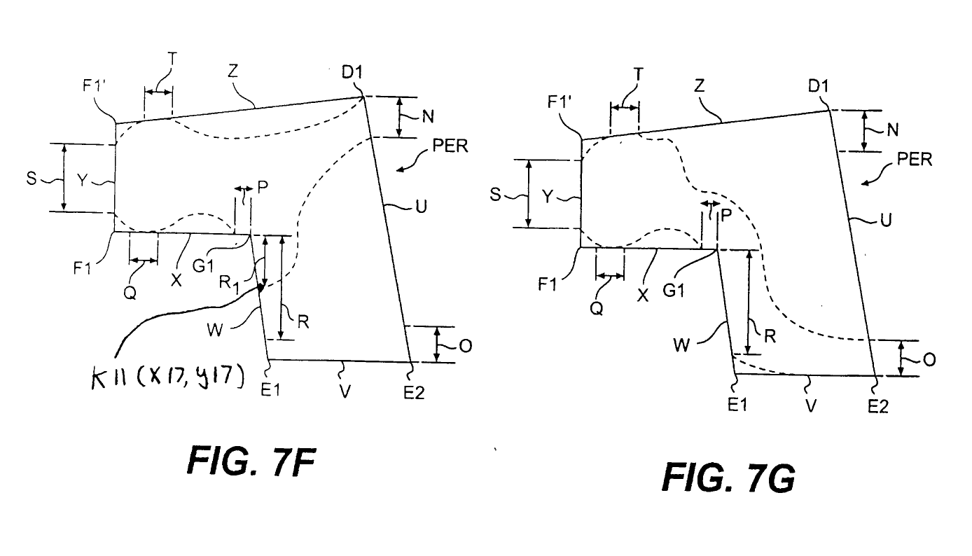

A rectangular measuring device 800 for measuring the hand 100, such as illustrated in FIG. 7A, can be used for determining sizes and shapes of handles or apparatus of the present invention, such as the forceps/tweezers handles or apparatus 400a, 400b. The rectangular measuring device 800 is desirably made of measuring members, such as four rulers, that can be used when the hand 100 is in the Forceps Hand Position (FHP) to measure sizes for the forceps/tweezers handles or apparatus of the present invention, such as forceps/tweezers handles or apparatus 400a and 400b. The rulers or measuring members are respectively indicated by the letters DD, EE, FF and GG, with ruler DD as a first measuring member, with ruler EE as a second measuring member, with ruler FF as a third measuring member and ruler GG as a fourth measuring member. Each ruler DD, EE, FF, GG is set at right angles to each other. Ruler DD and ruler EE are generally parallel and in the X-axis direction, as illustrated in FIG. 7A. Ruler FF and GG are generally parallel and in the Y-axis direction, as illustrated in FIG. 7A. Each ruler DD, EE, FF, GG has a corresponding slot 806, 807, 808, 809 along the center of its length and has corresponding gradation marks 805. The rulers DD, EE, FF and GG are connected by fastening members, such as rivets 801,802,803,804, or other desirable fasteners, such that the rulers DD, EE, FF and GG are loosely connected within the rectangular measuring device 800. The loose connection at rivets 801,802,803,804 allows each ruler DD, EE, FF, GG to slide along in the X-axis direction and/or the Y-axis direction. The dimensions, length and width, of Rulers DD, EE, and FF can generally be the same, whereas ruler GG can generally have a wider portion, such as a five millimeter wide segment 810, starting at elevation 812 at approximately half the length of ruler GG. The other half of ruler GG can be a standard ruler or other suitable measuring device and is identified as the narrow segment 811 of ruler GG. The wide segment 810 desirably has a wider width than the width of the narrow segment 811. Measurements start at the right end of ruler DD and EE indicated by RHT in FIG. 7A. Measurements start near ruler DD for ruler FF. Measurements start near ruler EE for ruler GG. The wide segment of Ruler GG faces the inside INS of the rectangular measuring device 800.

Referring to FIGS. 7A and 7B, the rectangular measuring device 800 is positioned along Plane C (see FIGS. 1B, 2, 6A and 6B) to measure a hand 100 in the Forceps Hand Position (FHP). The right end side RHT of ruler DD touches the horizontal crease 108 on the radial side 110 of the palm 102 of the hand 100. The right end side RHT of ruler EE is placed at point P1 in Area M of the palm 102 on the ulnar side 111 of the hand 100. As illustrated in FIG. 7A and FIG. 7B, ruler FF moves in the X-axis direction along ruler DD and in the X-axis direction along ruler EE until the outside edge FOUT of ruler FF is at the Plane B (see FIGS. 1A and 7B), where the tip 201a of the thumb 201 opposes the space 320 between the tip 200a of the index finger 202 and the tip 200a of the middle finger 203 on line 305. Ruler GG is moved in the X-axis direction along ruler DD and ruler EE until the narrow segment 811 of ruler GG touches the palmar surface 220 at the DIP joint 252 of the small finger 205. Ruler GG is then moved in the Y-axis direction until the elevation 812 on ruler GG contacts the radial side 110 of the DIP joint 252 of the small finger 205. The wide segment 810 now touches the palmar surface 210 of the ring finger 204. This completes positioning of rulers DD, EE, FF and GG for measuring a hand size.

Continuing with reference to FIGS. 7A, 7B, 7D and 7E, measurements are taken along measurement distances D, E, F, F′, G and H. Measurement distance D, as a first measurement distance, is measured on the outer side DOUT from the right end side RHT of ruler DD of the rectangular measuring device 800 from the horizontal crease 108 on the radial side 110 of the hand 100 to the outer side FOUT of ruler FF of the rectangular measuring device 800. Measurement distance E, as a second measurement distance, is measured along the inner side EIN of ruler EE on the rectangular measuring device 800 from the point P1 in area M on the ulnar side 111 of the hand 100 to the palmar surface 220 of the small finger 205, when the small finger 205 is touching the narrow segment 811 of ruler GG and the hand 100 is in the Forceps Hand Position (FHP). Measurement distance F, as a third measurement distance, is measured along the outer side FOUT of ruler FF on the rectangular measuring device 800 from the outer side DOUT of ruler DD to the ulnar side 203c of the middle finger 203 when the hand 100 is in the Forceps Hand Position (FHP). Measurement distance F′, as a fourth measurement distance, is measured along the outer side FOUT of ruler FF from the radial side 202c of the index finger 202 to the ulnar side 203c of the middle finger 203. Measurement distance G, as a fifth measurement distance, is measured along ruler GG on the inner side INS of the rectangular measuring device 800 from the inner side EIN of ruler EE to the radial side 217 of the ring finger 204 when the hand 100 is in the Forceps Hand Position (FHP). Measurement distance H, as a sixth measurement distance, is measured along ruler DD from the outer side FOUT of ruler FF to the wide segment 810 on the inner side GIN of ruler GG on the rectangular measuring device 800.

Continuing with reference to FIGS. 1A, 7A, 7B, 7D and 7E, measurement lines oriented and arranged to correspond to a hand in the Forceps Hand Position (FHP) are drawn from the corresponding first through sixth measurement distances D, E, F, F′, G and H and are desirably recorded on a medium such as on grid paper. These measurement lines are used to produce corresponding outline lines to outline a handle shape, with the outline formed by these outline lines for a handle shape being illustrated in FIGS. 7D and 7E, and also being indicated in FIG. 7B, such as for forceps/tweezers handles or apparatus 400a and 400b, of the present invention. First, a first measurement line corresponding to the second measurement distance E is drawn in the X-axis direction. Then, a second measurement line corresponding to the fifth measurement distance G is drawn in the Y-axis direction starting at a preselected distance, typically five millimeters (consistent with the elevation 812), to the left of the line drawn corresponding to the second measurement distance E. Next, a third measurement line corresponding to the sixth measurement distance H is drawn in the X-axis direction starting at the top GTOP of the second measurement line drawn for the fifth measurement distance G. Then a fourth measurement line corresponding to the third measurement distance F is drawn in the Y-axis direction. A fifth measurement line corresponding to the fourth measurement distance F′ is then drawn on the same fourth measurement line as the third measurement distance F. Next, a sixth measurement line corresponding to the first measurement distance D is drawn in the X-axis direction, and an end of the sixth measurement line for the first measurement distance D is on the same Y-axis direction line as the left end HLFT of the third measurement line for the sixth measurement distance H. Furthermore, the fourth measurement line for the third measurement distance F is drawn in the Y-axis direction with an end of the fourth measurement line starting at the left end DLFT of the sixth measurement line for the first measurement distance D.

As discussed above, a handle or apparatus, such as the forceps/tweezers handles or apparatus 400a and 400b, of the present invention can come in a plurality of sizes. The sizes of forceps/tweezers handles 400 of the present invention can be compiled by drawing point to point outline lines Z, Y, X, W, V and U to connect end points D1, F1′, F1, G1, E1 and E2 on the measurement lines corresponding to measurement distances D, E, F, F′, G and H as illustrated in FIGS. 7B, 7D and 7E. As illustrated in FIGS. 7A, 7B, 7D and 7E, the first point to point outline line is line Z, which connects end point D1 on the sixth measurement line corresponding to the first measurement distance D to end point F1′ on the fifth measurement line corresponding to the fourth measurement distance F′. The second point to point outline line is line Y connecting end point F1′ on the fifth measurement line corresponding to the fourth measurement distance F′ to end point F1 on the fourth measurement line corresponding to the third measurement distance F with the end point F1 also corresponding to the same end point on the third measurement line corresponding to the sixth measurement distance H. Then the third point to point outline line X connects end point F1 on the fourth measurement line corresponding to the third measurement distance F, which also corresponds to the same end point on the third measurement line corresponding to the sixth measurement distance H, to end point G1 on the second measurement line corresponding to the fifth measurement distance G, which is followed by the fourth point to point outline line W connecting end point G1 on the second measurement line corresponding to the fifth measurement distance G to end point E1 on the first measurement line corresponding to the second measurement distance E. The fifth point to point outline line V next connects end point E1 on the first measurement line corresponding to the second measurement distance E to end point E2 on the first measurement line corresponding to the second measurement distance E. Finally, the sixth point to point outline line U is drawn from end point D1 on the sixth measurement line corresponding to the first measurement distance D to end point E2 on the first measurement line corresponding to the second measurement distance E to complete the perimeter PER incorporating the outline lines Z, Y, X, W and V for measurements for handles or apparatus, such as the forceps/tweezers handles or apparatus 400a and 400b of the present invention. Perimeters PER for various hand sizes are compared to produce a range of sizes for handles of the present invention. As discussed above, the forceps/tweezers handles or apparatus 400a and 400b of the present invention can contact the hand 100 at the following locations: at the horizontal crease 108 of the palm 102 on the ulnar side 111 of the hand 100, at a location in area M of the palm 102 on the ulnar side 111 of the hand 100, the palmar surface 210 of the ring finger 204 with the palmar surface 220 of the small finger 205, at the radial side 110 of the ring finger 204, at the distal pad 201b of the thumb 201, at the distal pad 202b of the index finger 202 and at the distal pad 203c of the middle finger 203.

The sizes of the forceps/tweezers handles or apparatus, such as forceps/tweezers handles or apparatus 400a and 400b, of the present invention are determined by plotting or recording measurements of the perimeter PER. However, the shapes of the forceps/tweezers handles or apparatus, such as forceps/tweezers handles or apparatus 400a and 400b, of the present invention are related to those areas on the perimeter PER which potentially touch, or contact, the hand 100 at certain areas, dependent upon the particular use or application of the forceps/tweezers handles or apparatus, such as forceps/tweezers handles or apparatus 400a and 400b, of the present invention. Referring to FIGS. 7D through 7G, sections of various outline lines of the perimeter PER forming potential contact areas for handle or apparatus measurements are indicated by double-arrowed lines for corresponding contact areas with a hand 100.

Continuing with reference to FIGS. 7E through 7G, a first section N on the sixth outline line U extends approximately within a range from the point D1 to the point K1 on the sixth outline line U and is a first potential limited contact area on the perimeter PER for contacting an area of the hand related to the horizontal crease 108 of the palm 102 on the radial side 110 of the hand 100. A second section O on the sixth outline line U extends approximately within a range from the point E2 to the point K2 on the sixth outline line U and is a second limited potential contact area on the perimeter PER for contacting an area of the hand related to area M of the palm 102 on the ulnar side 111 of the hand 100. A third section R on the fourth outline line W extends approximately within a range from the point G1 to the point K3 on the fourth outline line W and is a third limited potential contact area on the perimeter PER for contacting an area of the hand related to the palmar surface 210 of the ring finger 204 and the palmar surface 220 of the small finger 205. A fourth section P on the third outline line X extends approximately within a range from the point G1 to the point K4 on the third outline line X and is a fourth limited potential contact area on the perimeter PER for contacting an area of the hand related to the radial side 217 of the ring finger 204.

A fifth section Q on the third outline line X extends approximately within a range from the point K5 to the point K6 on the third outline line X. A sixth section S on the second outline line Y extends approximately within a range from the point K7 to the point K8 on the second outline line Y. A seventh section T on the first outline line Z extends approximately within a range from the point K9 to the point K10 on the first outline line Z. The combination of a fifth section Q on the third outline line X, a sixth section S on the second outline line Y and a seventh section T on the first outline line Z are respectively fifth, sixth and seventh limited potential contact areas on the perimeter PER for contacting areas of the hand related to the distal pad 202b of the index finger 202 and the distal pad 203b of the middle finger 203. The combination of the fifth section Q on the third outline line X, the sixth section S on the second outline line Y and the seventh section T on the first outline line Z is duplicated on the distal ends 435a of the opposing blades 440 of a handle or apparatus, such as forceps/tweezers handles or apparatus 400a and 400b, of the present invention and can relate to an area for the distal pad 201b of the thumb 201.

Segments or sections of the respective sixth through first outline lines U, V, W, X, Y and Z that are not on the first section N on the sixth outline line U, the second section O on the sixth outline line U, the fourth section P on the third outline line X, the fifth section Q on the third outline line X, the third section R on the fourth outline line W, the sixth section S on the second outline line Y and the seventh section T on the first outline line Z on the perimeter PER can have any curve or shape because those areas generally do not contact parts of the hand 100 on a handle or apparatus, such as the forceps/tweezers handles or apparatus 400a and 400b, of the present invention, as long as the shape produced does not place pressure on the CT area.

Common contact sections on the perimeter PER for forceps/tweezers handles or apparatus of the present invention, such as forceps/tweezers handles or apparatus 400a and 400b, as discussed in reference to FIG. 7E, can include the fourth section P on the third outline line X, the fifth section Q on the third outline line X, the sixth section S on the second outline line Y and the seventh section T on the first outline line Z. Furthermore, forceps/tweezers handles or apparatus of the present invention, such as forceps/tweezers handles or apparatus 400a and 400b, can also share a portion of the third section R on the fourth outline line W. As illustrated in FIG. 7F, forceps/tweezers handle or apparatus 400a incorporates the section R1, which is a subsection of the section R, with the section R1 on the fourth outline line W extending approximately in a range from the point G1 to the point K11 on the fourth outline line W and the section R1 is a portion of the third limited potential contact area on the perimeter PER for contacting an area of the hand related to the palmar surface 210 of the ring finger 204. Whereas, as illustrated in FIG. 7G, the third limited potential contact area for forceps/tweezers handle or apparatus 400b extends approximately within the range of the entire length of the third section R on the fourth outline line W.

As evident from FIGS. 7E through 7G, forceps/tweezers handles or apparatus 400a and 400b do not share the same contact areas on the palm 102 of the hand 100. In the forceps/tweezers handle or apparatus 400a, the palmar contact area for forceps/tweezers handle or apparatus 400a is approximately located at the horizontal crease 108 at the radial side 110 of the palm 102 of the hand 100, which corresponds to the first section N on the sixth outline line U. Whereas in the forceps/tweezers handle or apparatus 400b, the palmar contact area for forceps/tweezers handle or apparatus 400b is approximately located at area M on the palm 102 on the ulnar side 111 of the hand 100, area M being approximately half the distance between the horizontal crease 108 and the pisiform bone 126, which corresponds to the second section O on outline line U.

As also illustrated in FIGS. 7F and 7G, the above described palmar contact areas determine a general shape of forceps/tweezers handle or apparatus 400a and a general shape of forceps/tweezers handle or apparatus 400b. As illustrated in FIG. 7F, as previously discussed, the forceps/tweezers handle or apparatus 400a of the present invention can be generally formed in the shape of an arc with extensions 422a near the middle of its convex sides 401. Whereas, as illustrated in FIG. 7G, the forceps/tweezers handle or apparatus 400b of the present invention can form a shape similar to a jog in the road with an entrance and an exit, with the road corresponding the outline shape of the handle or apparatus 400b and with the entrance and exit respectively corresponding to the sections N, O, and S on the perimeter PER of the forceps/tweezers handle or apparatus 400b.

Further, as illustrated in FIGS. 6A, 6B and 7B, the length L1 for forceps/tweezers handle or apparatus 400a is related to the linear distance from the proximal end 413a to the distal end 432a of forceps/tweezers handle or apparatus 400a with the hand 100 in the Forceps Hand Position (FHP). Length L1 can be measured along the x-axis in relation to the distance from point D1 at the right side RHT of ruler DD to line 305 where the tip 201a of the thumb 201 opposes the space 320 between the tip 200a of the index finger 202 and the tip 200a of the middle finger 203 on Plane B at the outer side FOUT of ruler FF of the rectangular measuring device 800. Furthermore, length L1 also relates to the distance from the horizontal crease 108 of radial side 110 of the hand 100 to tip 200a of the index finger 202 and tip 200a of the middle finger 203 taken with the hand 100 in the Forceps Hand Position (FHP). In addition, the length L2 for forceps/tweezers handle or apparatus 400b is in relation to the linear distance from the proximal end 413b to the distal end 432b of forceps/tweezers handle or apparatus 400b. Length L2 can be measured along the x-axis in relation to the distance from point E1 at the right side RHT of ruler EE to line 305 where the tip 201 a of the thumb 201 opposes the space 320 between the tip 200a of the index finger 202 and the tip 200a of the middle finger 203 on Plane B at the outer side FOUT of ruler FF of the rectangular measuring device 800. Length L2 also relates to the linear distance from point E2 in area M on the ulnar side 111 of the hand 100 to the index finger 202 and tip 200a of the middle finger taken with the hand in the Forceps Hand Position (FHP).

With reference to FIG. 7C, digital imaging can also be used as a medium for measuring a hand 100 to determine the length, slope, location and shape of the above described outline lines and sections on the perimeter PER and the configuration of the perimeter PER, such as when the hand 100 is in the Forceps Hand Position (FHP), to develop sizes for forceps/tweezers handles or apparatus, such as forceps/tweezers handles or apparatus 400a and 400b, of the present invention, similar to the sizes for handles for a apparatus developed by using the rectangular measuring device 800 of FIGS. 7A and 7B. As illustrated in FIG. 7C, such an image I can be produced by digital imaging of a hand 100 by an imaging system 700. The imaging system 700 can include a grid measuring system 701, a digital imaging apparatus 702, a processor 703, a display device 704 and a point indicator 705. The digital image I can be produced by the digital imaging apparatus 702, such as a digital image scanner or digital camera, that produces the digital image I which can be transferred by the processor 703, such as a computer, to the grid measuring device or grid measuring system 701, as can be displayed on the display device 704, such as a computer monitor, or printed by printer using a known computer or processing program, such as a Photoshop or other suitable imaging or graphics design program.

Similar to the discussion of FIGS. 7B and 7D through 7G, an imaging system, such as imaging system 700 of FIG. 7C, can be used to determine the locations of points D1, F1′, F1, G1, E1 and E2 and the length, slope, location, shape and configuration of sections N, 0, R, R1, P, Q, S and T on the perimeter PER, as well as the configuration of the perimeter PER, corresponding to similar locations and areas on the hand 100 used for determining sizes for the forceps/tweezers handles or apparatus of the present invention. These points D1, F1′, F1, G1, E1 and E2 and the sections N, 0, R, R1, P, Q, S and T can be determined from the image I on the grid measuring system 701 plotted using the processor 703 and the point indicator 705. The points and sections are selectively indicated on the grid measuring system 701 utilizing a point indicator 705, such as a computer mouse, in conjunction with a suitable processing program, typically with the hand 100 in Forceps Hand Position (FHP). Plotting of the points D1, F1′, F1, G1, E1 and E2 and the points corresponding to the sections N, 0, R, R1, P, Q, S and T on the perimeter PER determines the coordinates, such as X and Y axes coordinates (xn,yn) for the points D1, F1′, F1, G1, E1 and E2 and for the sections N, 0, R, R1, P, Q, S and T on the perimeter PER. Also, plotting of the points D1, F1′, F1, G1, E1 and E2 indicated by their corresponding coordinates generates the above described outline lines Z, Y, X, W, V and U, and determines the location, slope, length and configuration of these outline lines Z, Y, X, W, V and U, and connects these outline lines to form the Perimeter PER of a specific configuration for corresponding sizes of forceps/tweezers handles or apparatus of the present invention, such as forceps/tweezers handles or apparatus 400a and 400b.

As illustrated in FIG. 7C, as well as in FIGS. 7D and 7E, the point D1 (x1,y1) is consistent with the location of the radial side 110 of the horizontal crease 108 of the palm 102 of the hand 100. The point F1′ (x2,y2) is consistent with the location where the radial side 110 of the index finger 202 meets Plane B on line 305 where the tip 201a of the thumb 201 opposes the space 320 between the tip 200a of the index finger 202 and the tip 200a of the middle finger 203. The point F1 (x3,y3) is consistent with the location where the ulnar side 111 of the middle finger 203 meets Plane B on line 305 where the tip 201a of the thumb 201 opposes the space 320 between the tip 200a of the index finger 202 and the tip 200a of the middle finger 203. The point G1 (x4,y4) is consistent with and is the junction of the radial side 217 of the ring finger 204 with the palmar side 210 of the ring finger 204. The point E1 (x5,y5) is consistent with and is the junction of the palmar side 220 of the small finger 205 and the ulnar side 111 of the small finger 205. Finally, point E2 (x6,y6) is consistent with the location of point P1 in Area M on the ulnar side 111 of the palm 102 of the hand 100, point P1 being located on the ulnar 111 side of the palm 102 of the hand 100 approximately half way between the horizontal crease 108 and the pisiform bone 126 of the wrist 120. The coordinates of the points D1(x1,y1), F1′(x2,y2), F1(x3,y3), G1(x4,y4), E1(x5,y5) and E2(x6,y6) can then be sequentially connected, as illustrated in FIG. 7C through 7G, to form the outline lines Z, Y, X, W, V and U, as also illustrated in FIGS. 7B, which in turn form the Perimeter PER for a forceps/tweezers handle or apparatus of the present invention, such as forceps/tweezers handle or apparatus 400a, 400b.

As illustrated in FIG. 7C, the coordinates assigned to points on the perimeter PER using the image system 700 are consistent with the corresponding points in FIGS. 7D through 7G. The first section N on the sixth outline line U extends approximately within a range from the point D1 (x1,y1) to the point K1 (x7,y7) on the sixth outline line U. The second section O on the sixth outline line U extends approximately within a range from the point E2 (x6,y6) to the point K2 (x8,y8) on the sixth outline line U. The third section R on the fourth outline line W extends approximately within a range from the point G1 (x4,y4) to the point K3 (x9,y9) on the fourth outline line W; and the section R1 on the fourth outline line W extends approximately in a range from the point G1 (x4,y4) to the point K11 (x17,y17) on the fourth outline line W. The fourth section P on the third outline line X extends approximately within a range from the point G1 (x4,y4) to the point K4 (x10,y10) on the third outline line X. The fifth section Q on the third outline line X extends approximately within a range from the point K5 (x11,y11) to the point K6 (x12,y12) on the third outline line X. The sixth section S on the second outline line Y extends approximately within a range from the point K7 (x13,y13) to the point K8 (x14,y14) on the second outline line Y. The seventh section T on the first outline line Z extends approximately within a range from the point K9 (x15,y15) to the point K10 (x16,y16) on the first outline line Z.

Further, as an alternative measuring method and with reference to FIG. 7C1, digital imaging can also be used as a medium for measuring a hand 100 to determine the lengths L1 and L2 when the hand 100 is in the Forceps Hand Position (FHP). Such lengths L1 and L2 determined by digital imaging are similar to lengths L1 and L2 determined by using the rectangular measuring device 800 of FIGS. 7A and 7B. Such an image I can be produced by digital imaging of a hand 100 by the imaging system 700 including the display device 704. The digital image can be transferred to the grid measuring device or grid measuring system 701, as can be displayed on the display device 704, such as a computer monitor, or printed by printer using a suitable imaging or graphics design program.

Points D1, LL1, E2 and LL2, as illustrated in FIG. 7C1, can be placed on such an image I to determine lengths L1 and L2 for the forceps/tweezers handles or apparatus of the present invention. The point D1 (x1,y1) being consistent with the location of the radial side 110 of the horizontal crease 108 of the palm 102 of the hand 100. The point LL1 (x20,y20) being consistent with a position on line 305 on Plane B where the tip 201a of the thumb 201 opposes the space 320 between the tip 200a of the index finger 202 and the tip 200a of the middle finger 203, with the points LL1(x20,y20) and D1(x1,y1) being on a line Ln1 with the line Ln1 desirably being substantially parallel to the x-axis of the grid measuring system 701. The point E2 (x6,y6) being consistent with the location of point P1 in area M on the ulnar side 111 of the palm 102 of the hand 100, point P1 being located on the ulnar side 111 of the palm 102 of the hand 100 approximately half way between the horizontal crease 108 and the pisiform bone 126 of the wrist 120. The point LL2 (x21,y21) being consistent with a position on line 305 on Plane B where the tip 201a of the thumb 201 opposes the space 320 between the tip 200a of the index finger 202 and the tip 200a of the middle finger 203, with the points LL2(x21,y21) and E2(x6,y6) being on a line Ln2 with the line Ln2 also desirably being substantially parallel to the x-axis of the grid measuring system 701.

The distance of length L1 is then determined from measuring the distance on the plotted line Ln1 from point D1(x1,y1) to point LL1(x20,y20). Similarly the distance of length L2 is determined from measuring the distance on the plotted line Ln2 from point E2(x6,y6) to point LL2(x21,y21). Therefore, as illustrated in FIG. 7C1, the coordinates of the points D1(x1,y1) and LL1(x20,y20) are connected to measure the length L1 and the coordinates of the points E2(x6,y6), and LL2(x21,y21) are also connected to measure the length L2 for a forceps/tweezers handle or apparatus of the present invention, such as forceps/tweezers handle or apparatus 400a, 400b.

Variations of the Forceps/Tweezers Handle or Apparatus

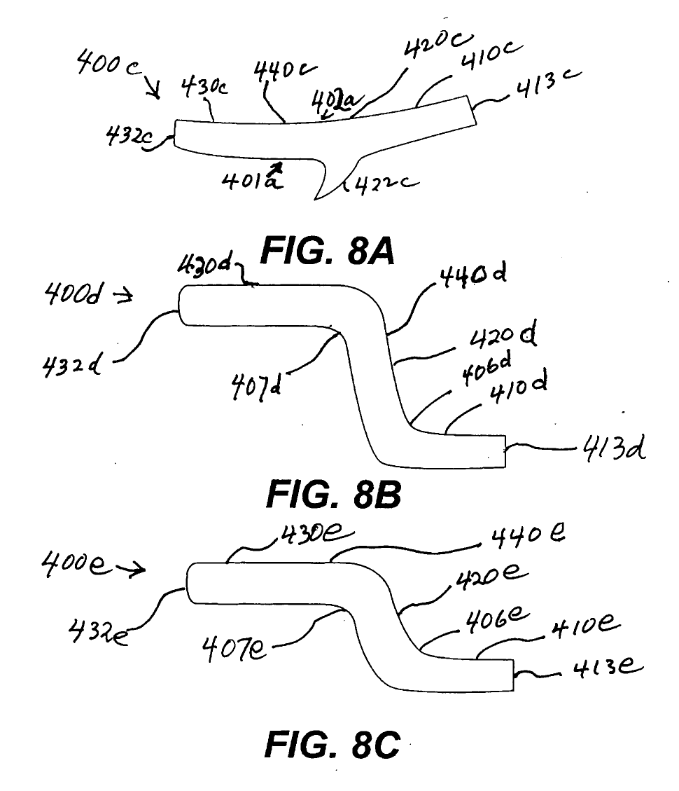

The forceps/tweezers handles or apparatus 400a and 400b of the present invention can have multiple variations. As illustrated in FIGS. 3A through 4B, distal ends 432a, 432b of the distal sections 430a, 430b of the preferred designs for forceps/tweezers handle 400a or forceps/tweezers handle 400b typically have adequate surface area to enable the thumb 201 to oppose the index finger 202 and the middle finger 203. Closing the wide distal ends 432a, 432b of the distal sections 430a, 440b of forceps/tweezers handles or apparatus 400a and 400b requires less effort to maintain pinch at the working ends of such a forceps/tweezers handle or apparatus when the thumb 201 opposes the index finger 202 and middle finger 203 than when the thumb 201 individually opposes either the index finger 202 or 203 middle finger. However, there can be circumstances when opposing the thumb 201 to either the index finger 202 or the middle finger 203 can be desirable while using a forceps/tweezers handle or apparatus of the present design. This condition is illustrated in the embodiments of FIGS. 8A, 8B and 8C of the present invention where the width of the distal ends 432c, 432d and 432e of the corresponding distal sections 430c, 430d and 430e of forceps/tweezers handles or apparatus 400c, 400d and 400e are less than the width Wad, Wbd of the corresponding distal ends 432a, 432b of the distal sections 430a, 430b of forceps/tweezers handles or apparatus 400a and 400b.

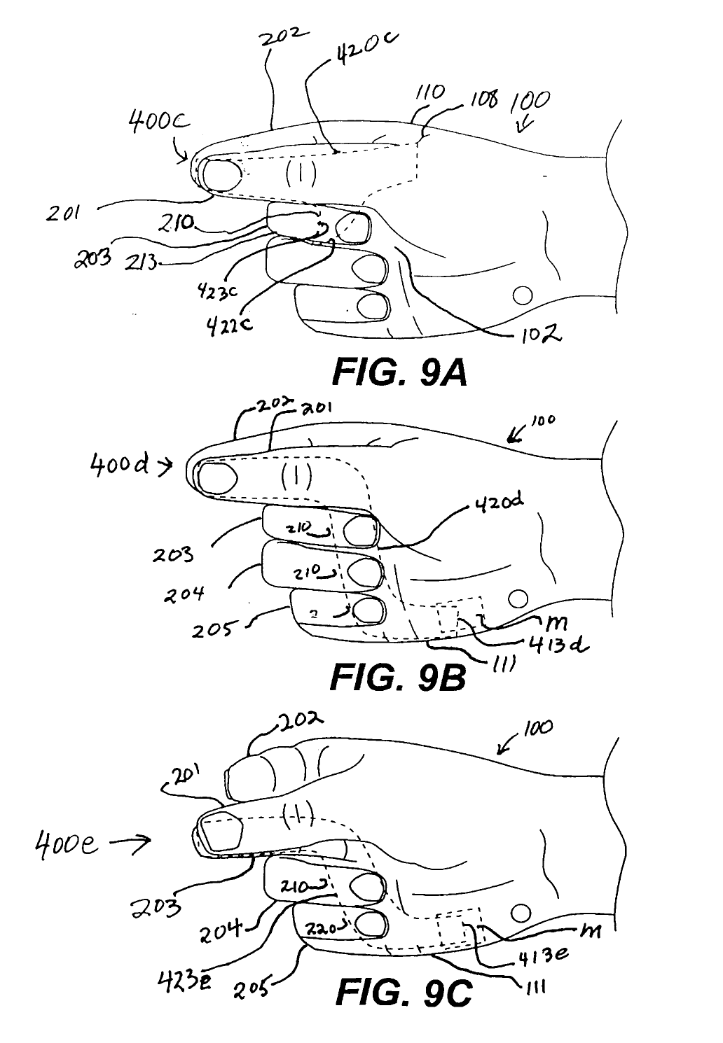

FIG. 8A illustrates a forceps/tweezers handle or apparatus 400c, which is similar to forceps/tweezers handle or apparatus 400a. The convex sides 401a, 401c of forceps/tweezers handles or apparatus 400a and 400c are arc shaped and share corresponding extensions 422a, 422c attached to their respective middle sections 420a, 420c. Forceps/tweezers handle or apparatus 400c can be formed by blades 440c or can have a unitary body, or an integrally formed body. As illustrated in FIG. 9A, the forceps/tweezers handle or apparatus 400c is supported and stabilized by contact and pull of the palmar surface 210 of the middle phalange 215 of the middle finger 203 on the distal surface 423c of the corresponding extensions 422c of the respective middle sections 420c of forceps/tweezers handle or apparatus 400c toward the radial side 110 of the horizontal crease 108 of the palm 102 of the hand 100. The forceps/tweezers handle or apparatus 400c also can be supported and stabilized by contact of the palmar surface 210 of the middle phalange 215 of the ring finger 204 at the distal surface 423c of the corresponding extensions 422c of the respective middle sections 420c to pull the forceps/tweezers handle or apparatus 400c toward the radial side 110 of the horizontal crease 108 of the palm 102 of the hand 100. Support with the ring finger 204 allows the index finger 202 to be free to manipulate a control mechanism for an implement that can be attached to forceps/tweezers handle or apparatus 400c. In contrast, forceps/tweezers handle or apparatus 400a has wide distal ends 432a instead of the narrow distal ends 432c of forceps/tweezers handle or apparatus 400c. The distal ends 432c of the distal sections 430c of the of the respective opposing blades 440c of the forceps/tweezers handle or apparatus 400c are closed by the opposing movement of the thumb 201 and the index finger 202 or by the opposing movement of the thumb 201 and the middle finger 203.

FIGS. 8B and 8C illustrates forceps/tweezers handles or apparatus 400d and 400e of the present invention which are similar to forceps/tweezers handle or apparatus 400b. Forceps/tweezers tweezers handles or apparatus 400b, 400d and 400e share a similar jogged shape configuration. The proximal sections 410b, 410d, 410e of forceps/tweezers handles or apparatus 400b, 400d and 400e have proximal curves 406b, 406d, 406e respectively leading into the middle sections 420b, 420d, 420e. The middle sections 420b, 420d, 420e of forceps/tweezers handles or apparatus 400b, 400d and 400e have distal curves 407b, 407d, 407e respectively extending into the distal sections 430b, 430d, 430e. However, forceps/tweezers handles or apparatus 400d and 400e can be formed by blades 440d, 440e or can have a unitary body, or integrally formed body. Also, in contrast, the distal ends 432b of the distal sections 430b of forceps/tweezers handle or apparatus 400b are relatively wide, whereas the distal ends 432d, 432e of the corresponding distal sections 430d, 430e of the respective forceps/tweezers handles or apparatus 400d and 400e are relatively narrow.

FIG. 9B illustrates a hand holding forceps/tweezers handle or apparatus 400d with the thumb 201 opposing the index finger 202, while the palmar surface 210 of the middle phalange 215 of the middle finger 203, the palmar surface 210 of the middle phalange 216 the ring finger 204 and the palmar surface 220 of the middle phalange 225 of the small finger 205 contact the corresponding distal surface 423d of the respective middle sections 420d for support and stabilization, while also pulling the proximal end 413d of the proximal section 410d of the forceps/tweezers handle or apparatus 400d toward Area M on the ulnar side 111 of the hand 100.

FIG. 9C illustrates a hand holding forceps/tweezers handle or apparatus 400e with the thumb 201 opposing the middle finger 203 and with the palmar surfaces 210 of the middle phalange 216 of the ring finger 204 and the palmar surface 220 of the middle phalange 225 of the small finger 205 contacting the corresponding distal surfaces 423e of the respective middle sections 420e for support and stabilization, while pulling the proximal end 413e of the proximal end 410e of the forceps/tweezers handle or apparatus 400e toward area M on the ulnar side 111 of the hand 100. Furthermore, in the embodiment of the forceps/tweezers handle or apparatus 400e, support with the ring finger 204 and small finger 205 allows the index finger 202 to be free to manipulate a control mechanism for an implement that can be attached to forceps/tweezers handle or apparatus 400e.

FIGS. 10A, 10B and 10C illustrate variations to forceps/tweezers handles or apparatus 400a and 400c where the distal surfaces 423a, 423c of the middle sections 420a, 420c meet the palmar surface 210 of the middle phalange 215 of the ring finger 205. FIG. 10A illustrate scalloped distal surface 424a, 424c, FIG. 10B illustrates a generally flat distal surface 425a, 425c, and FIG. 10C illustrates a ringed distal surface 426a, 426c of the corresponding extensions 422a, 422c of the respective middle sections 420a, 420c. FIGS. 10D and 10E illustrate variations to the corresponding distal surfaces 423b, 423d, 423e of the middle sections 420b, 420d, 420e of the respective forceps/tweezers handles or apparatus 400b, 400d and 400e, with FIG. 10D showing scalloped variations 424b, 424d, 424e and including a step portion 427b, 427d 427e for positioning the ring finger 204 and small finger 205, and with FIG. 10E showing a flat distal surface 425b, 425d, 425e of the middle sections 420b, 420d, 420e of the forceps/tweezers handles or apparatus 400b, 400d, 400e of the present invention. As illustrated in FIG. 10C, rings, or other suitable positioning devices, can be employed to position a corresponding long finger that is used in supporting the tweezers/forceps handles or apparatus 400b, 400d and 400e.

Handles or apparatus, such as the forceps/tweezers handles or apparatus 400a, 400b, 400c, 400d and 400e, of the present invention can be made in one size or various sizes based on above described measurements with reference to FIGS. 7A through 7G. As illustrated in FIGS. 11A and 11B, an alternative to making multiple sizes of the various embodiments of forceps/tweezers handles or apparatus of the present invention, such as forceps/tweezers handles or apparatus 400a and 400b, is to add extensions 490a, 490b to adapt the forceps/tweezers handles or apparatus 400a and 400b of the present invention to a range of hand sizes. As shown in FIG. 11A, for example, an extender 490a can be added to the proximal end 413a of the proximal section 410a of the forceps/tweezers handle or apparatus 400a. Likewise, as illustrated in FIG. 11B, an extender 490b can be added to the proximal end 413b of the proximal section 410b of the forceps/tweezers handle 400b of the present invention.

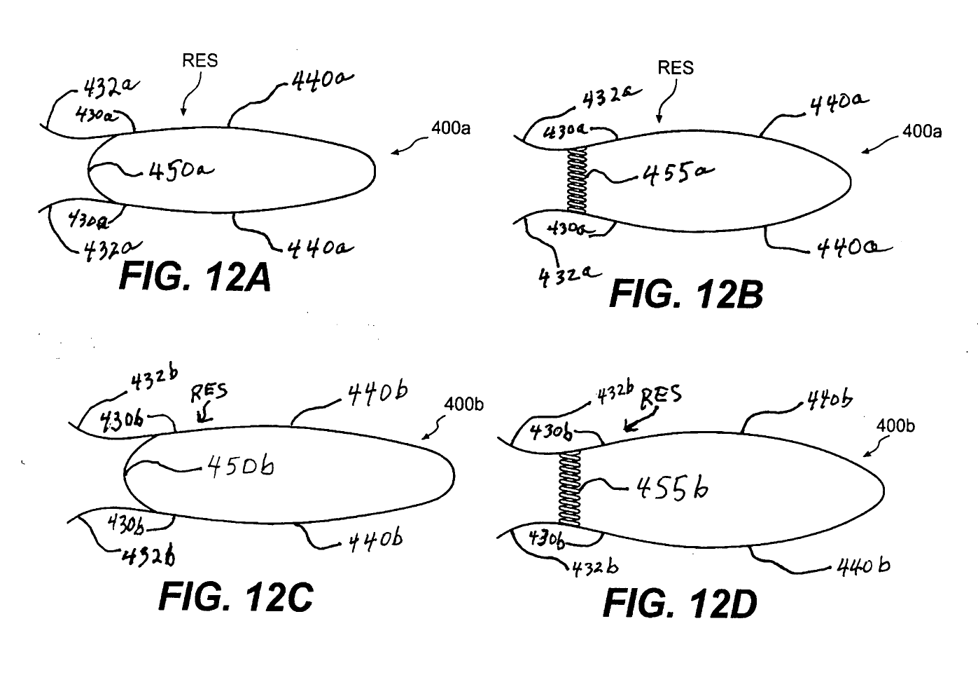

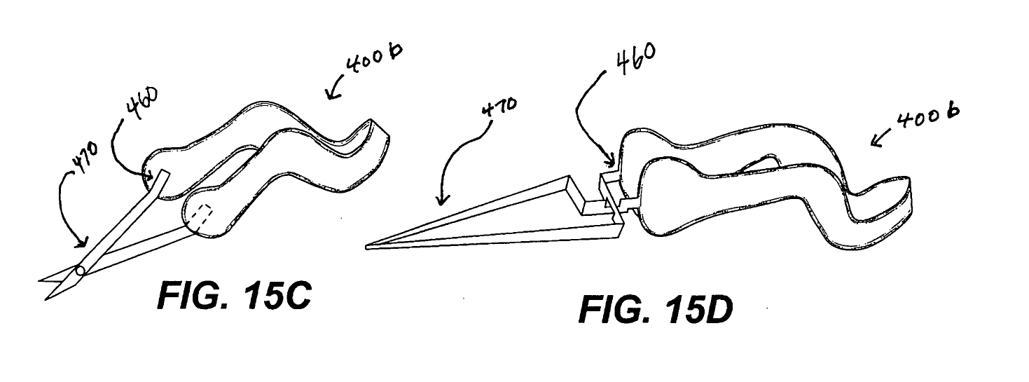

It can be desirable when pinch is relaxed to maintain a resting distance at the opposing blades 440a, 440b between the distal ends 432a, 432b of the distal sections 430a, 430b of forceps/tweezers handles or apparatus 400a and 400b. FIGS. 12A, 12B, 12C and FIG. 12D illustrate a flat spring member 450a, 450b and a coiled spring member 455a, 455b inserted between distal ends 432a, 432b of the corresponding distal sections 430a, 430b of the respective opposing blades 440a, 440b to maintain a resting position RES for the forceps/tweezers handles or apparatus 400a and 400b of the present invention. The flat spring member 450a, 450b as shown in FIGS. 12A and 12C can also maintain alignment of the distal ends 432a, 432b of the distal sections 430a, 430b to limit drift of integrated or attached working ends 470 with respect to each other as illustrate in FIGS. 14 through 15D.

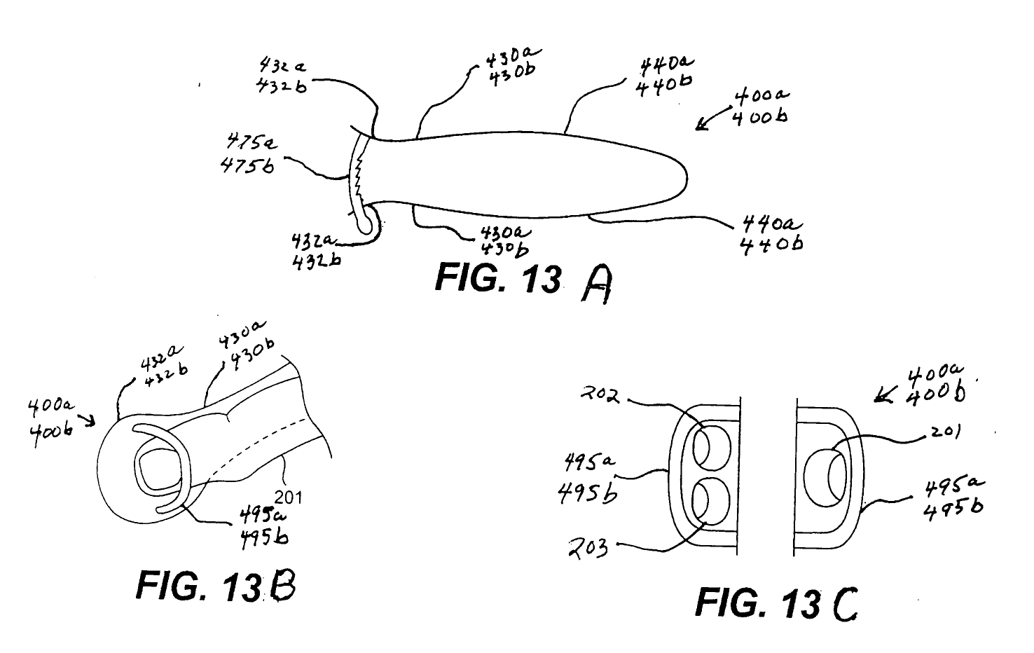

FIGS. 13A illustrates a clamping mechanism 475a, 475b inserted between distal ends 432a, 432b of the distal sections 430a, 430b of the opposing blades 440a, 440b to maintain a closed or partially closed position for a handle or apparatus, such as forceps/tweezers handles or apparatus 400a and 400b, of the present invention.

In another variation, as shown in FIGS. 13B and 13C, finger guide members 495a 495b for the thumb 201, index finger 202 and middle finger 203 can be attached to the distal ends 432a, 432b of the distal sections 430a, 430b of the respective opposing blades 440a, 440b of a handle or apparatus, such as the forceps/tweezers handles or apparatus 400a and 400b of the present invention, to spread the distal ends 432a, 432b of the forceps/tweezers handles or apparatus 400a and 400b of the present invention.

FIGS. 14 illustrates a connection means 460 which can be placed to join or integrate a working end 470 at the distal ends 432a, 432b of the corresponding distal sections 430a, 430b of the respective opposing blades 440a, 440b of forceps/tweezers handles or apparatus 400a and 400b of the present invention. The connection means 460 illustrated in FIG. 14 has a guide sleeve 462 to assist engaging and removing the attaching part 464 of the working end 470 in a spring-like mechanism 466 in order to fix or hold the attaching part 464 of the working end 470 to the distal ends 432a, 432b of the corresponding distal sections 430a, 430b of the respective forceps/tweezers handles or apparatus 400a and 400b of the present invention. Such a connection means 460 can maintain the position of the working end 470 in forceps/tweezers handles or apparatus 400a and 400b of the present invention so as to allow the tips 472 of two similar working ends 470 to meet without drift. Furthermore, such a connecting means 460 can allow the exchange of various types of working ends 470 in handles or apparatus of the present invention.

FIGS. 15A and 15B illustrate working ends 470 attached to a forceps/tweezers handle or apparatus 400a of the present invention, with the working end 470 of FIG. 15A being a microscissors and the working end 470 of FIG. 15B being a reverse tweezers, and with each corresponding working end 470 being connected by a suitable connection means 460 to a forceps/tweezers handle or apparatus 400a. FIGS. 15C and 15D illustrate working ends 470 attached to a forceps/tweezers handle 400b of the present invention, with the working end 470 of FIG. 12C being a microscissors and the working end 470 of FIG. 12D being a reverse tweezers, and with each corresponding working end 470 being connected by a suitable connection means 460 to a forceps/tweezers handle or apparatus 400b.

FIGS. 16A and 16B illustrate unitary handle or apparatus embodiments 400f and 400g of the present invention. Unitary handle or apparatus 400f has a unitary blade 490f in a single arc shape configuration with an extension 422f at the middle section 420f, similar to forceps/tweezers handle or apparatus 400a, and unitary handle 400g has a unitary blade 490g in a shape similar to a jog in the road shape, or “jogged” shaped, similar to forceps/tweezers handle or apparatus 400b. However, unitary handles or apparatus 400f and 400g, respectively, have the unitary blade 490f and the unitary blade 490g instead of a pair of opposing blades 440a and 440b as illustrated for forceps/tweezers handles or apparatus in FIGS. 3A through 4B. The unitary handle or apparatus 400f engages the corresponding portions of the hand 100 as previously described relating to FIGS. 5A and 6A for the forceps/tweezers handle or apparatus 400a of the present invention. Furthermore, the unitary handle 400g engages corresponding portions of the hand 100 as previously described relating to FIGS. 5B and 6B for the forceps/tweezers handle or apparatus 400b of the present invention.

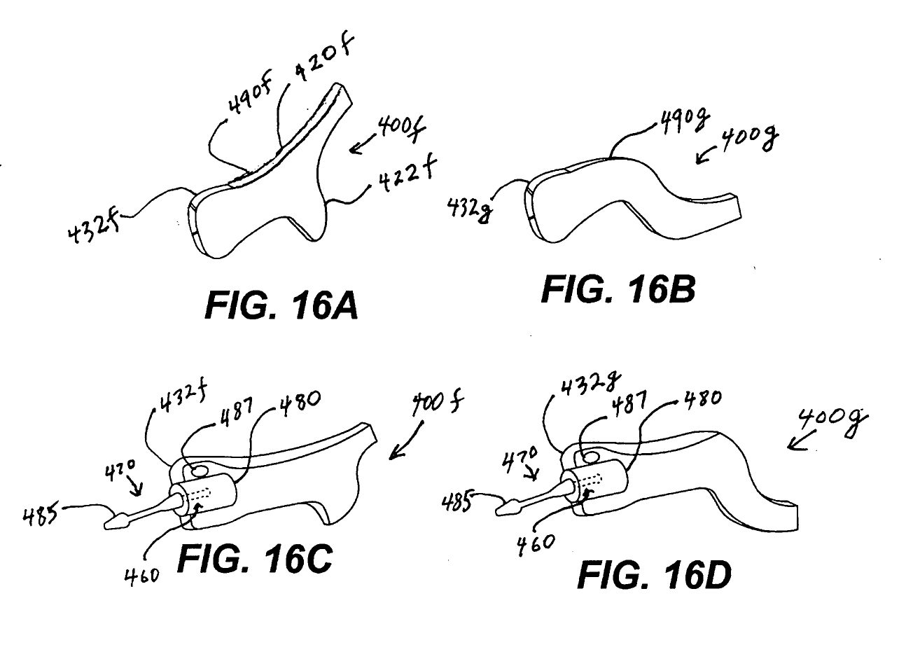

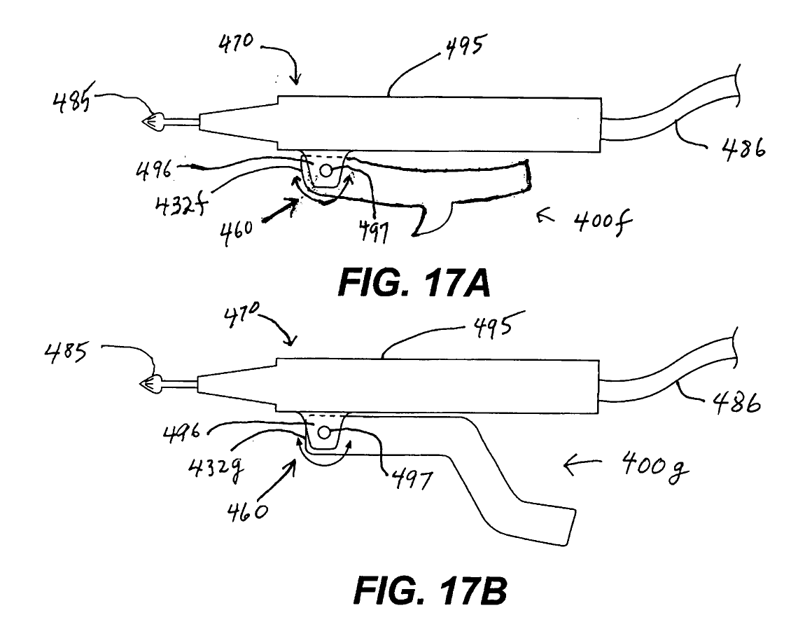

In reference to FIGS. 16A, 16B, 17A and 17B unitary handles 400f and 400g of the present invention can have various attached replaceable or integrated devices and implements. Examples of attached replaceable or integrated devices and implements can include embodiments of the unitary handles 400f and 400g that incorporate a motor driving means 480 for rotation or movement of a working end 470 or an implement 485, such as an attachable drill bit or screwdriver, as illustrated in FIGS. 16C and 16D.

Further, FIGS. 17A and 17B illustrate an implement 495 attached to unitary handles or apparatus 400f and/or 400g of the present invention. A source for power can be included or attached by cable 486 to conduct power to the implement 495. Controlling devices 487, as illustrated in FIGS. 16C and 16D, can be integrated with implement 495 or with the distal ends 432f, 432g of respective unitary handles or apparatus 400f and 400g. The controlling devices 487 can be manipulated, such as by the thumb, index finger or middle finger or by the combined action of the thumb opposing the index or middle finger, so that the thumb side and long finger side can oppose each other and squeeze a switch or controlling device. In addition, a plurality of different types of controlling devices 487 can be attached at multiple areas on and in relation to the forceps/tweezers handles or apparatus 400c, 400d and 400e of the present invention illustrated in FIGS. 8A through 9C.

As illustrated in FIGS. 17A and 17B, the implement 495, such as a dental drill, can be incorporated with corresponding unitary handles 400f and/or 400g of the present invention at hinge member 496. Such hinge member 496 can rotate about shaft 497 for changing the angle of working end 470 of the implement 495 relative to the position of the hand 100 holding unitary handles or apparatus 400f or 400g of the present invention. Also, examples of implements that can be attached to the unitary handles or apparatus 400f and 400g can include small power drills, paint brushes, glue guns and other implements that act like a stylus to score or mark a surface.

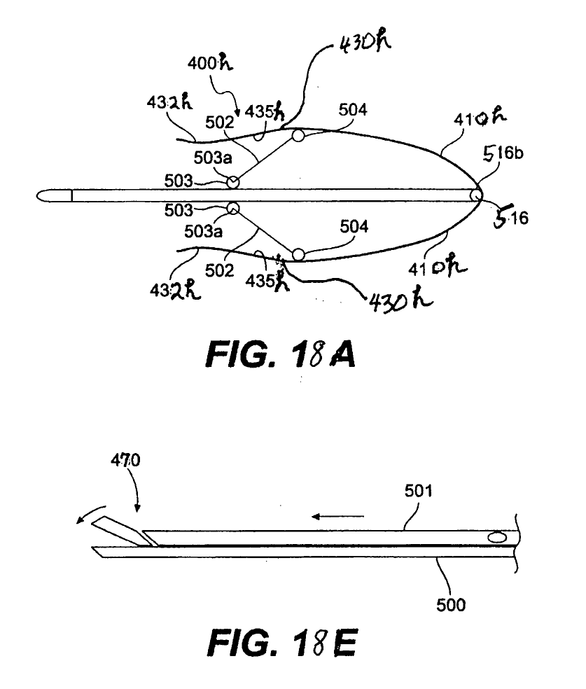

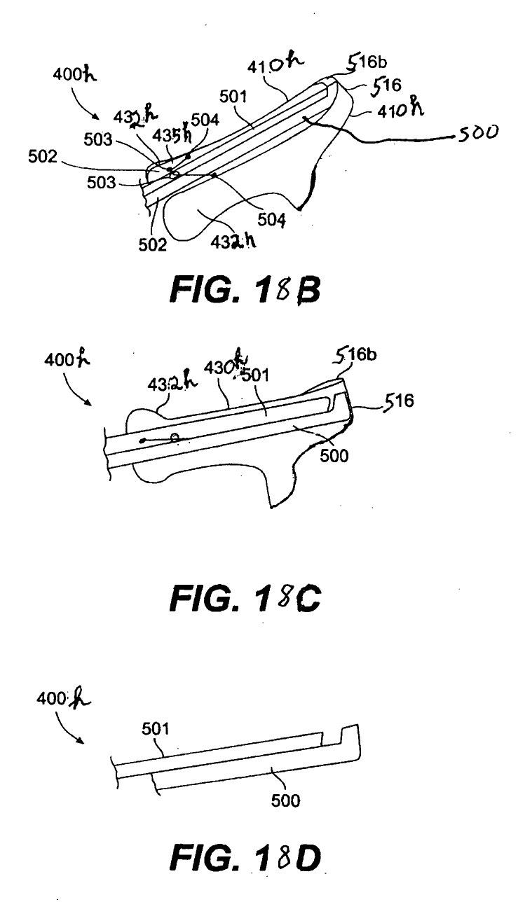

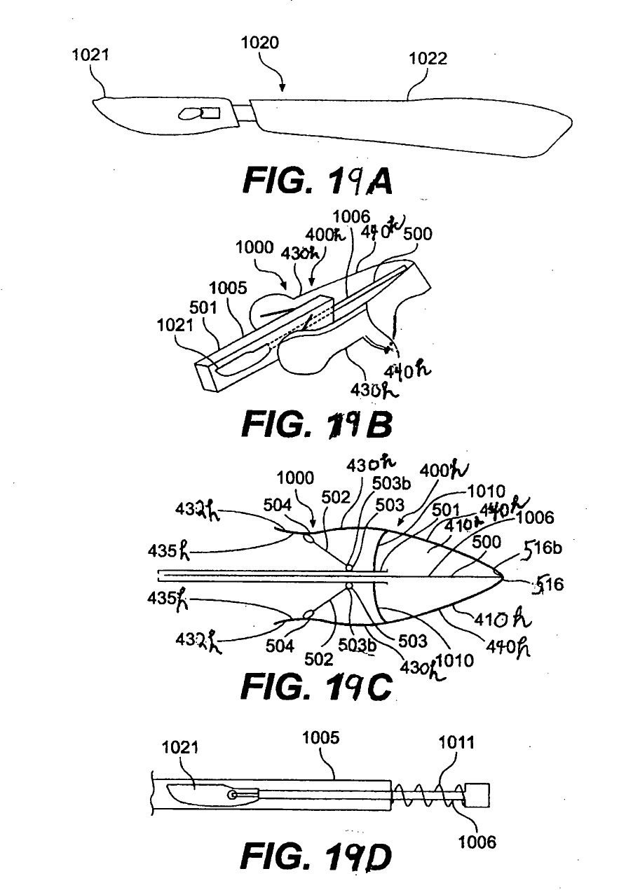

Forceps/tweezers apparatus 400h, as illustrated in FIG. 18A, FIG. 18B, FIG. 18C, FIG. 18D, FIG. 18E, FIG. 19A and FIG. 19B is a variation of the forceps/tweezers handle and apparatus 400a of the present invention that changes the direction of motion at the working ends 470 of forceps/tweezers handle and apparatus 400a of the present invention. Usually, the direction of movement at the tips of a common forceps is generally parallel to the opposing motion of the thumb 201 to the index finger 202 and middle finger 203. A pituitary rongeur is a common example of a surgical instrument, for cutting or biopsy of tissue, used with an up and down, instead of a side-to-side opening and closing motion. Opening and closing the ring handles of the pituitary rongeur position the thumb 201 and index finger 202 or middle finger 203 of the hand 100 in a proximal and distal relationship to each other. Proximal-distal motion activates a sliding member to move or activate the working members to open and close. Using such an instrument in which the thumb 201 and a long finger 200 of the hand 100 move in a proximal/distal direction typically is not as comfortable for the hand 100 as using the side-to-side opposing motion utilized in common forceps or tweezers.

However, the working ends 470 of forceps/tweezers handle and apparatus 400h of the present invention, as illustrated in FIG. 18A, FIG. 18B, FIG. 18C, and FIG. 19B, can convert the opposing (side-to-side) motion of the thumb 201 to the index finger 202 and the middle finger 203 to a slanted or vertical motion in relation to opposing thumb 201, index finger 202 and middle finger 203 of the hand 100. The forceps/tweezers handle or apparatus 400h has a fixed member 500 attached to the inside 516b of the radial hinge 516 of the proximal section 410h of the forceps/tweezers handle or apparatus 400h of the present invention. Above the fixed member 500 is a sliding member 501 that activates the working ends 470 to open and close as illustrated in FIG. 18E. Brace members 502 connect the sliding member 501 to the inside aspect 435h of the distal ends 432h of the distal sections 430h. Sliding member hinges 503 attach the ends 503a of the brace members 502 to the sliding members 501 and distal end hinges 504a attach to the inner aspect 435h of the distal ends 432h of the distal sections 430h of variation holder 400h of the present invention. Pinching the distal ends 432h of the distal sections 430h of the forceps/tweezers handle and apparatus 400h of the present invention moves the braces 502 at the hinges 503, 504 to move the sliding member 501. In addition, the fixed member 500 can have a rotating mechanism at the inside 516b of the radial hinge 516 for rotation of the working ends 470.

As illustrated in FIGS. 18A and 18B, squeezing the distal ends 432h of the distal sections 430h of the forceps/tweezers handle or apparatus 400h of the present invention, when the sliding member hinges 503 of the braces 502 are placed distal to hinges 504, the sliding member 501 moves away from the hand 100. Furthermore, as illustrated in FIGS. 19B and 19C, squeezing the distal ends 432h of the distal sections 430h of forceps/tweezers handle or apparatus 400h of the present invention, when the sliding member hinges 504 of the braces 502 are placed proximal to hinges 503b on sliding member 501, moves the sliding member 501 toward the hand 100. Moving the sliding member 501 can actuate any of a variety of mechanisms attached to the end of the sliding member 501, such as to close a fine scissors, other implement or working ends 470, such as illustrated in FIG. 18E. In addition, with reference to FIGS. 18A through 18E, the forceps/tweezers handle or apparatus 400h of the present invention can also be utilized in conjunction with various endoscopic or surgical tools, as well as other types of working tools that work at a distance from the operator.

Continuing with reference to FIGS. 19A through 19D, the standard scalpel 1020, illustrated in FIG. 19A, is in the shape of a stylus and is a fixture in surgery. During the course of surgery, a scalpel blade 1021 can contact pathogens harboring in the patient's serum. One problem associated with the standard scalpel handle 1022 is penetrating wounds to operating room personnel. Inadvertent sharp wounds can transmit diseases to the assisting personnel. An automatic retractable blade guard could prevent sharp wounds.

As previously discussed, forceps/tweezers handle or apparatus 400h of the present invention can move a sliding member 501 in relation to a fixed member 500. FIG. 19B illustrates a retractable scalpel 1000 with a retractable sliding guard 1005 that surrounds a fixed scalpel member 1006 attached to scalpel blade 1021. FIG. 19C illustrates a flat spring member 1010 attached to the opposing blades 440h and the retractable sliding guard 1005. Alternately, a coil spring member 1011 can be attached to the fixed scalpel member 1006 and the retractable sliding guard 1005 as shown in FIG. 19D. When the distal ends 432h of the distal sections 430h of the opposing blades 440h of the fixed scalpel member 1006 of forceps/tweezers handle or apparatus 400h of the present invention are squeezed, the brace members 502 move the proximal hinges 503 to direct the retractable sliding guard 1005 toward the hand of an operator to expose the scalpel blade 1021. When the distal ends 432h of the distal sections 430h are released, the retractable sliding guard 1005 automatically covers the scalpel blade 1201.

One advantage of the retractable scalpel 1000 with the forceps/tweezers handle variation handle 400h of the present invention is promoting protection from sharp injury in the operating room. Another advantage is that the retractable scalpel 1000 is based on the anatomic Forceps Hand Position (FHP), which can make the retractable scalpel 1000 more comfortable for the hand 100 to hold and manipulate.

In summary, handles or apparatus of a design according to the present invention can be molded or formed into and contiguous with any of many types of equipment commonly held by a hand. Furthermore, handles or apparatus based on the design method of the present invention can be attached to or integrated into objects that can be lifted, rotated, moved, carried, etc. Such handles or apparatus of the present invention can advantageously be attached or integrated into or with an object or working end. Additionally, such handles or apparatus of the present invention can be designed to swivel and/or rotate on various axes at a location of attachment. For example, the handle or apparatus can be attached to a shaft by an extension member, such as for turning.

Also, in the handles or apparatus of the present invention, various materials can be used for fabrication of the handles or apparatus as, for example, various woods, metals, plastics, composites, rubber compounds, latex's and organic or inorganic materials, suitable for the particular application of a handle or apparatus of the present invention. Further, various materials can be added to augment and personalize the fit of a handle or apparatus of the present invention.

The principles, preferred embodiments and modes of operation of the present invention have been described in the foregoing specification. However, the invention which is intended to be protected is not limited to the particular embodiments disclosed. The embodiments described herein are illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1.) A forceps type apparatus for use with a hand, comprising:
a proximal section, the proximal section for engaging one of a portion of the radial side of the palmar surface of the hand or the ulnar side of the palmar surface of the hand without placing substantial pressure on a surface of the hand located over the carpal tunnel;
a middle section, the middle section connecting the proximal section with a distal section, with the middle section for engaging at least one the middle finger, ring finger or small finger of the hand; and
a distal section, the distal section extending from the middle section for receiving the thumb and at least one of the index finger or middle finger of the hand, when the forceps type apparatus is positioned with the hand.

2.) The forceps type apparatus of claim 1, wherein the distal section includes a working end.

3.) The forceps type apparatus of claim 2, wherein the working end includes an implement.

4.) The forceps type apparatus of claim 3, wherein the implement is for grasping, pinching, cutting, rotating, an electrical function or a mechanical function.

5.) The forceps type apparatus of claim 1, wherein the proximal section positions the forceps type apparatus within the hand without engaging a surface of the hand located over the carpal tunnel.

6.) The forceps type apparatus of claim 1, wherein the forceps type apparatus includes a pair of opposing blades.

7.) The forceps type apparatus of claim 6, wherein the pair of opposing blades each comprise a proximal section, a middle section and a distal section.

8.) The forceps type apparatus of claim 7, wherein each of the pair of opposing blades are a mirror image of the other of the pair of opposing blades.

9.) The forceps type apparatus of claim 7, wherein the distal section of the corresponding opposing blades includes a working end.

10.) The forceps type apparatus of claim 9, wherein the working end is located at the distal end of the distal section of the forceps type apparatus.

11.) The forceps type apparatus of claim 10, wherein the working end is for at least one of grasping, pinching or cutting.

12.) The forceps type apparatus of claim 9, wherein the working end performs a mechanical action related to the opposing movement of the distal pad of the thumb on one opposing blade toward the distal pad of at least one of the index finger and the distal pad of the middle finger on the other opposing blade.

13.) The forceps type apparatus of claim 9, wherein the working end of at least one of the opposing blades has a suitable connection means.

14.) The forceps type apparatus of claim 9, wherein the working end of at least one of the opposing blades has a suitable connection means for connecting an implement to the forceps type apparatus.

15.) The forceps type apparatus of claim 9, wherein the working end of a corresponding opposing blade is located at a distal end of the of at least one of the opposing blades has a suitable connection means for connecting an implement to the forceps type apparatus.

16) The forceps type apparatus of claim 9, wherein an implement is integrated with the working end of at least one of the opposing blades.

17.) The forceps type apparatus of claim 9, wherein the implement is for grasping, pinching, cutting, rotating, an electrical function or a mechanical function.

18.) The forceps type apparatus of claim 7, wherein the proximal section, the middle section and the distal section form a generally arc shaped configuration.

19.) The forceps type apparatus of claim 18, wherein the generally arc shaped configuration forms a generally concave side at a top side and an generally convex side at a bottom side for the forceps type apparatus.

20.) The forceps type apparatus of claim 19, wherein the convex side includes an extension from a corresponding middle section of a corresponding opposing blade of the forceps type apparatus.

21.) The forceps type apparatus of claim 18, wherein the proximal sections of each opposing blade meet and are connected by a radial hinge at the proximal end of the forceps type apparatus.

22.) The forceps type apparatus of claim 21, wherein the radial hinge comprises a mechanical connection means

23.) The forceps type apparatus of claim 18, wherein the middle section includes an extension from a corresponding opposing blade of the forceps type apparatus.

24.) The forceps type apparatus of claim 7, wherein the proximal section, the middle section and the distal section of a corresponding opposing blade form a generally jogged shape configuration.

25.) The forceps type apparatus of claim 24, wherein the generally jogged shape configuration includes a generally straight entrance configuration at the proximal end of the proximal section and a generally straight exit configuration at the distal end of the distal section.

26) The forceps type apparatus of claim 24, wherein the proximal sections of each opposing blade meet and are connected by an ulnar hinge at the proximal end of the forceps type apparatus.

27.) The forceps type apparatus of claim 26, wherein the ulnar hinge comprises a mechanical connection means

28.) The forceps type apparatus of claim 7, wherein the proximal sections of each opposing blade are continuous with or integrally formed into the other opposing blade.

29.) The forceps type apparatus of claim 7, wherein the width of the proximal end of the corresponding opposing blade approximates the width of base of the index finger of the hand.

30.) The forceps type apparatus of claim 7, wherein the distal end of the distal section of the corresponding opposing blade approximates the combined width of the distal pad of the index finger and the distal pad of the middle finger of the hand.

31.) The forceps type apparatus of claim 7, wherein the proximal ends of the proximal sections the corresponding opposing blades of the forceps type apparatus correspond to the surface of the palm in the area of the horizontal crease at the radial side of the hand.

32.) The forceps type apparatus of claim 7, wherein the proximal ends of the proximal sections of the corresponding opposing blades of the forceps type apparatus correspond to an area on the palmar surface of the hand approximately half the distance between the horizontal crease and the pisiform bone on the ulnar side of the palm of the hand

33.) The forceps type apparatus of claim 1, wherein the width of the proximal end of the proximal section of the forceps type apparatus approximates the width of base of the index finger of the hand.

34.) The forceps type apparatus of claim 1, wherein the distal end of the distal section of the forceps type apparatus approximates the combined width of the distal pad of the index finger and the distal pad of the middle finger of the hand.

35.) The forceps type apparatus of claim 1, wherein the proximal end of the proximal section of the forceps type apparatus corresponds to the surface of the palm in the area of the horizontal crease at the radial side of the hand.

36.) The forceps type apparatus of claim 1, wherein the proximal end of the proximal section of the forceps type apparatus corresponds to an area on the surface of the palm of the hand approximately half the distance between the horizontal crease and the pisiform bone on the ulnar side of the palm of the hand

37.) The forceps type apparatus of claim 1, wherein the proximal section, the middle section and the distal section form a unitary structure.

38.) The forceps type apparatus of claim 1, wherein the proximal section, the middle section and the distal section form a unitary structure including a unitary blade.

39.) The forceps type apparatus of claim 38, wherein the unitary blade includes a working end.

40.) The forceps type apparatus of claim 39, wherein the working end includes an implement.

41.) The forceps type apparatus of claim 40, wherein the implement is for grasping, pinching, cutting, rotating, an electrical function or a mechanical function.

42.) The forceps type apparatus of claim 38, wherein the unitary blade includes an implement.

43.) The forceps type apparatus of claim 42, wherein the implement is for grasping, pinching, cutting, rotating, an electrical function or a mechanical function.

44.) A handle for use with a hand, comprising:
a proximal section, the proximal section for engaging one of a portion of the radial side of the palmar surface of the hand or the ulnar side of the palmar surface of the hand without placing substantial pressure on a surface of the hand located over the carpal tunnel;
a middle section, the middle section connecting the proximal section with a distal section, with the middle section for engaging at least one the middle finger, ring finger or small finger of the hand; and
a distal section, the distal section extending from the middle section for receiving the thumb and at least one of the index finger or middle finger of the hand, when the handle is positioned with the hand.

45.) The handle of claim 44, wherein the distal section includes a working end.

46.) The handle of claim 45, wherein the working end includes an implement.

47.) The handle of claim 46, wherein the implement is for grasping, pinching, cutting, rotating, an electrical function or a mechanical function.

48.) The handle of claim 44, wherein the distal section includes an implement.

49.) The handle of claim 48, wherein the implement is for grasping, pinching, cutting, rotating, an electrical function or a mechanical function.

50.) The handle of claim 44, wherein the proximal section positions the handle within the hand without engaging a surface of the hand located over the carpal tunnel.

51.) The handle of claim 44, wherein the middle section placing substantial pressure on the surface of the hand located over the carpal tunnel.
